(19) United States
(12) Patent Application Publication
(10) Pub. No.: US 2011/0150878 A1
(43) Pub. Date: Jun. 23, 2011

(54) COMPOSITIONS AND METHODS FOR MANIPULATING LEVELS OF ANTIGEN-SPECIFIC ANTIBODIES IN A MAMMAL

(75) Inventor: Arpad Z. BARABAS, Calgary (CA)

(73) Assignee: University Technologies International, Incorporated, Calgary (CA)

(21) Appl. No.: 13/036,633

(22) Filed: Feb. 28, 2011

Related U.S. Application Data
(63) Continuation of application No. 10/568,127, filed on Aug. 16, 2006.
(60) Provisional application No. 60/495,804, filed on Aug. 15, 2003.

(51) Int. Cl. A61K 39/395; A61P 37/00

(52) U.S. Cl. 424/136.1

(57) ABSTRACT

The invention provides compositions and methods for increasing the levels of an autoantigen-specific IgM antibody in a mammal and, thus, decreasing the levels of a circulating autoantigen in a mammal. Using these autoantigen-specific IgM antibodies, the invention provides compositions and methods for ameliorating an autoimmune disease in a mammal. In one aspect, the invention provides compositions and methods for increasing the levels of an antigen-specific IgG antibody in a mammal and, thus, decreasing the levels of a circulating antigen in a mammal. Using these antigen-specific IgG antibodies, the invention provides compositions and methods for ameliorating a disease or condition in a mammal, e.g., a cancer or a foreign antigen, such as a pathogen.

### FIELD OF THE INVENTION

This invention relates to the fields of immunology and medicine. In one aspect, the invention provides compositions and methods for increasing the levels of an autoantigen-specific IgM antibody in a mammal and, thus, decreasing the levels of a circulating autoantigen in a mammal. Using these autoantigen-specific IgM antibodies, the invention provides compositions and methods for ameliorating an autoimmune disease in a mammal. In one aspect, the invention provides compositions and methods for increasing the levels of an antigen-specific IgG antibody in a mammal and, thus, decreasing the levels of a circulating antigen in a mammal. Using these antigen-specific IgG antibodies, the invention provides compositions and methods for ameliorating a disease or condition in a mammal, e.g., a cancer or a foreign antigen, such as a pathogen.

### BACKGROUND

Autoimmunity implies some reactivity of immune system components with normal or abnormal self. One of the most important functions of the immune system is to remove cell debris derived from the continuously damaged cells. If intracytoplasmic high molecular weight (MW) substances from the continuously damaged cells were allowed to accumulate in the circulation, toxicity and/or pathogenic autoantibody response(s) against self could result. The products of the CD5+ cells, autoantigen-specific IgM, can assist in the removal/catabolism of intracytoplasmic autoantigens to help maintain tolerance to self. Naturally occurring IgM antibodies are involved in the removal of tissue breakdown products. Specific circulating IgM anti-tissue antibodies have been observed in humans in disease conditions where cellular breakdown occurs, e.g. anti-heart antibodies in patients with myocardial infarction, in certain liver diseases and subsequent to burn injury. Thus, a restricted form of immune response consisting of IgM antibodies specific for particulate subcellular components can exist in the normal individual.

Cryptic autoantigens can be exposed to the immune system following cell death, e.g., as a result of toxic damage, hypoxia etc. Cryptic autoantigens can be liberated into tissue spaces, blood, urine, gut, etc., where they can initiate an IgM antibody response and subsequently be removed and/or catabolized. If cryptic autoantigens are modified as a result of being exposed to a modifying agent (chemical, toxic, infectious agent etc.) then these modified autoantigens will be recognized as foreign and pathogenic IgG response will follow. This can result in direct injury to a target organ or indirect injury by immune complexes, e.g., made up of the modified/unmodified antigens and pathogenic IgG antibodies settling into the glomeruli, other blood vessels, corrective tissues and the like.

### SUMMARY

The invention provides methods and compositions for increasing the levels of an autoantigen-specific IgM antibody in a mammal comprising the following steps: (a) providing a composition comprising an unmodified autoantigen and an antigen-specific multi-valent antibody, wherein the multi-valent antibody is specific for the autoantigen and is native to the mammal or is non-immunogenic to the mammal, and the autoantigen is present in the composition in molar excess to the multi-valent antibody; and (b) administering to the mammal an amount of the composition sufficient to increase the levels of the antigen-specific IgM antibody in the individual. The invention provides methods and compositions for decreasing the levels of a circulating autoantigen in a mammal comprising the following steps: (a) providing a composition comprising an unmodified autoantigen and an antigen-specific multi-valent antibody, wherein the multi-valent antibody is specific for the autoantigen and is native to the mammal or is non-immunogenic to the mammal, and the autoantigen is present in the composition in molar excess to the multi-valent antibody; (b) administering to the mammal an amount of the composition sufficient to increase the levels of an antigen-specific IgM antibody in the individual, thereby decreasing the levels of circulating autoantigen in the mammal. The invention provides methods and compositions for ameliorating an autoimmune disease in a mammal comprising the following steps: (a) providing a composition comprising an unmodified autoantigen and an antigen-specific multi-valent antibody, wherein the multi-valent antibody is specific for the autoantigen and is native to the mammal or is non-immunogenic to the mammal, and the autoantigen is present in the composition in molar excess to the multi-valent antibody; and (b) administering to the mammal an amount of the composition sufficient decrease the levels of the circulating autoantigen in the mammal, thereby ameliorating the autoimmune disease in the mammal. In alternative aspects, by ameliorating an autoimmune disease the methods can treat, lessen the severity of, slow or prevent the onset of, and/or slow the progress of the autoimmune disease. In one aspect, the mammal is a human.

In alternative aspects, the multi-valent antibodies used in the methods and compositions of the invention comprise entities with tri-valency, 4-valency, penta- or more valency. In one aspect, the multi-valent antibody comprises an IgM. In alternative aspects, the multi-valent antibody comprises multi-antigen-binding portions, i.e., multi-antigen binding sites, including, multi-fragments, subsequences, complementarity determining regions (CDRs) that retain capacity to bind antigen, including multi-(i) Fab fragments, monovalent fragments consisting of the VL, VH, CL and CH1 domains; (ii) F(ab′)2 fragments, bivalent fragments comprising two Fab fragments linked by a disulfide bridge at the hinge region; (iii) Fd fragments consisting of the VH and CH1 domains; (iv) Fv fragments consisting of the VL and VH domains of a single arm of an antibody, (v) dAb fragments (Ward et al., (1989) Nature 341:544-546), which consists of a VH domain; and/or (vi) isolated complementarity determining regions (CDRs). In one aspect, the multi-valent antibody comprise multi-single chain antibodies.

In one aspect, the multi-valent antibodies used in the methods and compositions of the invention comprise an isolated antibody, a synthetically generated antibody or a recombinantly generated antibody. The multi-valent antibody can comprise a chimeric antibody, e.g., a humanized antibody. In one aspect, the multi-valent antibody comprises a human antibody generated in a transgenic mouse. The transgenic mouse can comprise a human immunoglobulin gene locus. Multi-valent antibodies used in any of the methods or compositions of the invention can include human antibodies generated by a transgenic non-human animal, such as a mouse, capable of producing human antibodies, as described by, e.g., U.S. Pat. Nos. 5,939,598; 5,877,397; 5,874,299; 5,814,318.

In one aspect, in making the composition comprising the autoantigen and the antigen-specific multi-valent antibody, the unmodified autoantigen is mixed with the multi-valent antibody immediately before administration, or, the unmodified autoantigen is mixed with the multi-valent antibody between about 1 minute and two hours, or more, before administration, or, the autoantigen is mixed with the multi-valent antibody between about 5 minutes and one hour before administration, or, the autoantigen is mixed with the multi-valent antibody between about 10 minutes and 30 minutes before administration.

In one aspect, in making the composition comprising the autoantigen and the antigen-specific multi-valent antibody, the unmodified autoantigen is mixed with the multi-valent antibody and the mixture is freeze-dried. The freeze-dried mixture can be reconstituted in a formulation for administration at the time of administration. The freeze-dried mixture can be stored at a temperature of between about −20° C. and 4° C. The freeze-dried mixture can be reconstituted in an aqueous formulation, such as sterile distilled water or buffered saline, e.g., PBS, Ringer's and the like.

In one aspect, the autoantigens used in the methods and compositions of the invention comprise a purified autoantigen. The autoantigen can comprise a recombinant or synthetic polypeptide. The autoantigen can comprise a soluble antigen or a particulate antigen. The autoantigen can comprise a small molecular weight antigen, e.g., having a molecular weight between about 0.1 to 10 kd or about 0.5 to 5 kd, or, the autoantigen can comprise a large molecular weight antigen, e.g., a molecular weight of between about 5 to 50 kd or about 10 to 25 kd.

In one aspect, the autoantigens used in the methods and compositions of the invention comprise an autoantigen involved in an autoimmune response. The autoantigen can comprise a kidney tubular nephritogenic antigen, a glomerular nephritogenic antigen, an endometrial repro-EN-1.0 antigen, an endometrial IB1 antigen, glutamic acid decarboxylase, nucleolar ASE-1 antigen, Ro/SSA, La/SSB, nRNP, Sm, transaldolase, myelin basic protein, 70 kD mitochondrial biliary autoantigen, human cartilage glycoprotein 39, human Sp17 protein or human placental Hp-8. Autoantigens used in the methods and compositions of the invention can further comprise a plurality of autoantigens involved in the autoimmune response.

In one aspect, the autoantigen comprises a subcellular fraction, a cell, a tissue or an organ involved in the autoimmune response. The cell or the tissue can comprise a subcellular fraction, a cell or tissue homogenate or a cell, tissue or organ extract. In one aspect, the subcellular fraction, cell, tissue or organ comprises renal proximal tubules or renal proximal convoluted tubules or subcellular fractions thereof. The autoimmune response can comprise an autoimmune response to a kidney glomerular basement membrane autoantigen or a renal proximal convoluted tubule antigen.

In one aspect, the autoimmune disease comprises an autoimmune kidney disease, such as passive Heymann nephritis, lupus nephritis or membranous nephropathy. In alternative aspects, the autoimmune disease comprises rheumatoid arthritis, myasthenia gravis, endometriosis, autoimmune insulin-dependent diabetes mellitus (IDDM), systemic lupus erythematosus (SLE), Sjogren's syndrome, autoimmune hypoparathyroidism, multiple sclerosis (MS), primary biliary cirrhosis (PBC), autoimmune hemolytic anemia, contact sensitivity dermatitis, autoimmune blistering disorders (e.g., pemphigus vulgaris, pemphigus foliaceus, bolus pemphigoid), autoimmune infertility, autoimmune Addison's disease, myasthenia gravis, autoimmune thyroiditis or scleroderma.

In one aspect, there is anywhere between about 1% and 1000% more autoantigen present on a molar basis in the composition than multi-valent antibody. For example, in alternative aspects, there is about 1%, 2%, 3%, 4%, 5%, 6%, 7%, 8%, 9%, 10%, 20%, 25%, 30%, 35%, 40%, 45%, 50%, 55%, 60%, 65%, 70%, 75%, 80%, 85%, 90%, 95%, 100%, 125%, 150%, 175% or 200% more autoantigen present on a molar basis in the composition than multi-valent antibody. In one aspect, an alternative formulation comprising multi-valent antibody and antigen on an equimolar basis can be used in practicing the invention. In some aspects, maintenance dosages of formulation only need an equimolar formulation of multi-valent antibody and antigen.

In alternative aspects, pharmaceutical compositions of the invention and compositions used in the methods of the invention can comprise between about 1 μgm and 500 mg, or more, of antigen and an appropriate amount of antibody (bi-valent or multivalent) to keep the antigen in molar excess to the antibody. In other aspects, pharmaceutical compositions of the invention and compositions used in the methods of the invention can comprise between about 0.1 mg to 10 mg, or, 0.1 mg to 1.0 mg of antigen and an appropriate amount of antibody to keep the antigen in molar excess to the antibody. In one aspect, the composition comprises between about 0.2, 0.3, 0.4, 0.5, 0.6, 0.7, 0.8 or 0.9 mg of antigen and an appropriate amount of antibody to keep the antigen in molar excess to the antibody. In one aspect, the antibody (bi-valent or multivalent) used in a method or composition of the invention has a known titer (for antigen).

In alternative aspects, pharmaceutical compositions of the invention and compositions used in the methods of the invention can be administered by any route, e.g., parenterally, orally, intranasally or by an ocular route. In one aspect, the composition is administered once a day, twice a day, or three times a day. The composition can be administered about once to twice a week. The composition can be administered initially twice a week for about three weeks, then weekly for about five months, then monthly. The composition can comprise a sterile aqueous formulation. In one aspect, once the immune system is tuned to respond to the injected complexes of the invention, then injection of autoantigen alone can also maintain the specific immune response (though at a lower immune response level).

The invention provides pharmaceutical compositions comprising (i) an unmodified autoantigen and an antigen-specific multi-valent antibody, wherein the multi-valent antibody is specific for the autoantigen and is native to the mammal or is non-immunogenic to the mammal, and the autoantigen is present in the composition in molar excess to the multi-valent antibody, and (ii) a pharmaceutically acceptable excipient. In alternative aspects, the multi-valent antibodies used in the pharmaceutical compositions and methods of the invention comprise entities with tri-valency, 4-valency, penta- or more valency. In one aspect, the multi-valent antibody comprises an IgM. In alternative aspects, the multi-valent antibody comprises multi-antigen-binding portions, i.e., multi-antigen binding sites, including, multi-fragments, subsequences, complementarity determining regions (CDRs) that retain capacity to bind antigen, including multi-(i) Fab fragments, monovalent fragments consisting of the VL, VH, CL and CH1 domains; (ii) F(ab′)2 fragments, bivalent fragments comprising two Fab fragments linked by a disulfide bridge at the hinge region; (iii) Fd fragments consisting of the VH and CH1 domains; (iv) Fv fragments consisting of the VL and VH domains of a single arm of an antibody, (v) dAb fragments (Ward et al., (1989) Nature 341:544-546), which consists of a VH domain; and/or (vi) isolated complementarity determining regions (CDRs). In one aspect, the multi-valent antibody comprise multi-single chain antibodies. In one aspect, the multi-valent antibodies used in the pharmaceutical compositions and methods of the invention comprise an isolated antibody, a synthetically generated antibody or a recombinantly generated antibody. The multi-valent antibody can comprise a chimeric antibody, e.g., a humanized antibody. In one aspect, the multi-valent antibody comprises a human antibody generated in a transgenic mouse. The transgenic mouse can comprise a human immunoglobulin gene locus. Multi-valent antibodies used in any of the pharmaceutical compositions or methods of the invention can include human antibodies generated by a transgenic non-human animal, such as a mouse, capable of producing human antibodies, as described by, e.g., U.S. Pat. Nos. 5,939,598; 5,877,397; 5,874,299; 5,814,318. In one aspect, the multi-valent antibody comprises an IgM. In one aspect, the multi-valent antibody comprises an isolated antibody, a synthetically generated antibody or a recombinantly generated antibody. In one aspect, the multi-valent antibody used in the pharmaceutical compositions of the invention comprises an humanized antibody.

The invention provides pharmaceutical compositions, and methods of making them, made by a process comprising mixing an unmodified autoantigen with a multi-valent antibody immediately before administration. In one aspect, the unmodified autoantigen is mixed with the multi-valent antibody between about 1 minute and two hours before administration, or, the autoantigen is mixed with the multi-valent antibody between about 5 minutes and one hour before administration, or, the autoantigen is mixed with the multi-valent antibody between about 10 minutes and minutes before administration.

The invention provides pharmaceutical compositions, and methods of making them, made by a process comprising freeze-drying or lyophilized a mix of autoantigen and antigen-specific multi-valent antibody. The mix can be a fresh mix, or, as discussed above, an amount of time (a delay) can pass before the unmodified autoantigen is and multi-valent antibody mixture is freeze-dried or lyophilized. The freeze-dried mixture can be reconstituted in a formulation for administration at the time of administration. The freeze-dried mixture can be stored at a temperature of between about −20° C. and 4° C. The freeze-dried mixture can reconstituted in an aqueous formulation, e.g., sterile distilled water or buffered saline and the like.

The invention provides pharmaceutical compositions comprising a purified or isolated autoantigen, or, an autoantigen comprising a recombinant or synthetic polypeptide. The autoantigen can comprise a soluble antigen or a particulate antigen, or, a small molecular weight antigen, e.g., having a molecular weight (MW) of between about 0.1 to 10 kd or about 0.5 to 5 kd, or the autoantigen can comprise a large molecular weight antigen, e.g., having a MW of between about 5 to 50 kd or about 10 to 25 kd.

The invention provides pharmaceutical compositions comprising an autoantigen involved in an autoimmune response. The autoantigen can be any known autoantigen, or, a new autoantigen, which can be determined using routine screening methods. In alternative aspects, the autoantigen comprises a kidney glomerular basement membrane autoantigen, a kidney tubular nephritogenic antigen, a glomerular nephritogenic antigen, an endometrial repro-EN-1.0 antigen, an endometrial B31 antigen, glutamic acid decarboxylase, nucleolar ASE-1 antigen, Ro/SSA, La/SSB, nRNP, Sm, transaldolase, myelin basic protein, 70 kD mitochondrial biliary autoantigen, human cartilage glycoprotein 39, human Sp17 protein, human placental Hp-8.

In one aspect, pharmaceutical compositions of the invention can further comprise a single autoantigen, or, a plurality of different autoantigens involved in one or more autoimmune responses. In one aspect, the autoantigen comprises a subcellular fraction, a cell, a tissue or an organ involved in the autoimmune response. In one aspect, the cell or the tissue comprises a subcellular fraction, a cell or tissue homogenate or a cell, tissue or organ extract. The subcellular fraction, cell, tissue or organ can comprise renal proximal tubules or renal proximal convoluted tubules or subcellular fractions thereof. The autoimmune response can comprises an autoimmune response to a kidney glomerular basement membrane autoantigen or a renal proximal convoluted tubule antigen. The autoimmune response comprises an autoimmune kidney disease, such as passive Heymann nephritis, lupus nephritis or membranous nephropathy. The autoimmune response can comprise any autoimmune disease, for example, rheumatoid arthritis, myasthenia gravis, endometriosis, autoimmune insulin-dependent diabetes mellitus (IDDM), systemic lupus erythematosus (SLE), Sjogren's syndrome, autoimmune hypoparathyroidism, multiple sclerosis (MS), primary biliary cirrhosis (PBC), autoimmune hemolytic anemia, contact sensitivity dermatitis, autoimmune blistering disorders (e.g., pemphigus vulgaris, pemphigus foliaceus, bolus pemphigoid), autoimmune infertility, autoimmune Addison's disease, myasthenia gravis, autoimmune thyroiditis, scleroderma.

In one aspect of pharmaceutical compositions of the invention there is about 10%, 20%, 25%, 30%, 35%, 40%, 45%, 50%, 55%, 60%, 65%, 70%, 75%, 80%, 85%, 90%, 95%, 100%, 125%, 150%, 175% or 200% more autoantigen present on a molar basis in the composition than multi-valent antibody. Pharmaceutical compositions of the invention can comprise between about 1 μgm and 500 mg, or more, of antigen and an appropriate amount of antibody to keep the antigen in molar excess to the antibody. In other aspects, pharmaceutical compositions of the invention and compositions used in the methods of the invention can comprise between about 0.1 mg to 10 mg, or, 0.1 mg to 1.0 mg of antigen and an appropriate amount of antibody to keep the antigen in molar excess to the antibody. In one aspect, the composition comprises between about 0.2, 0.3, 0.4, 0.5, 0.6, 0.7, 0.8 or 0.9 mg of antigen and an appropriate amount of antibody to keep the antigen in molar excess to the antibody.

In one aspect, the pharmaceutical compositions of the invention are formulated to be administered by any route, e.g., parenterally, orally, intranasally or by an ocular route. In one aspect, pharmaceutical compositions of the invention are administered once a day, twice a day, or three times a day. In one aspect, the pharmaceutical compositions of the invention are administered about once to twice a week. In one aspect, the pharmaceutical compositions of the invention are administered initially twice a week for about three weeks, then weekly for about five months, then monthly.

In one aspect, the pharmaceutical compositions of the invention are formulated as a sterile liquid formulation, e.g., sterile saline, PBS, Ringer's and the like, for, e.g., injection, infusion, spraying, and the like.

The invention provides methods for increasing the levels of an antigen-specific IgG antibody in a mammal comprising the following steps: (a) providing a composition comprising a modified antigen and an antigen-specific bi-valent antibody, wherein the bi-valent antibody is specific for the antigen and is native to the mammal or is non-immunogenic to the mammal, and the modified antigen is present in the composition in molar excess to the bi-valent antibody; and (b) administering to the mammal an amount of the composition sufficient to increase the levels of the antigen-specific IgG antibody in the individual. The invention provides methods for decreasing the levels of a circulating antigen in a mammal comprising the following steps: (a) providing a composition comprising a modified antigen and an antigen-specific bi-valent antibody, wherein the bi-valent antibody is specific for the antigen and is native to the mammal or is non-immunogenic to the mammal, and the modified antigen is present in the composition in molar excess to the bi-valent antibody; (b) administering to the mammal an amount of the composition sufficient to increase the levels of an antigen-specific IgG antibody in the individual, thereby decreasing the levels of the circulating antigen in the mammal. The invention provides methods for ameliorating a disease or condition in a mammal comprising the following steps: (a) providing a composition comprising a modified antigen and an antigen-specific bi-valent antibody, wherein antigen is associated with the disease or condition, the bi-valent antibody is specific for the antigen and is native to the mammal or is non-immunogenic to the mammal, and the modified antigen is present in the composition in molar excess to the bi-valent antibody; and (b) administering to the mammal an amount of the composition sufficient increase the level of antigen-specific bi-valent antibody in the mammal, thereby ameliorating the disease or condition in the mammal. The invention provides pharmaceutical compositions comprising (i) a modified antigen and an antigen-specific bi-valent antibody, wherein the bi-valent antibody is specific for the antigen and is native to the mammal or is non-immunogenic to the mammal, and the modified antigen is present in the composition in molar excess to the bi-valent antibody, and (ii) a pharmaceutically acceptable excipient. In one aspect, the mammal is a human.

In alternative aspects, the bi-valent antibodies used in the methods and compositions of the invention comprises an IgG or an IgA. In alternative aspects, the bi-valent antibody comprises two antigen-binding portions, i.e., bi-valent antigen binding sites, including, bi-valent fragments, subsequences, complementarity determining regions (CDRs) that retain capacity to bind antigen, including bi-valent (i) Fab fragments, monovalent fragments consisting of the VL, VH, CL and CH1 domains; (ii) F(ab′)2 fragments, bivalent fragments comprising two Fab fragments linked by a disulfide bridge at the hinge region; (iii) Fd fragments consisting of the VH and CH1 domains; (iv) Fv fragments consisting of the VL and VH domains of a single arm of an antibody, (v) dAb fragments (Ward et al., (1989) Nature 341:544-546), which consists of a VH domain; and/or (vi) isolated complementarity determining regions (CDRs). In one aspect, the antibodies comprise bi-valent single chain antibodies.

In one aspect, the bi-valent antibodies used in the methods and compositions of the invention comprise an isolated antibody, a synthetically generated antibody or a recombinantly generated antibody. The bi-valent antibody can comprise a chimeric antibody, e.g., a humanized antibody. In one aspect, the bi-valent antibody comprises a human antibody generated in a transgenic mouse. The transgenic mouse can comprise a human immunoglobulin gene locus. Bi-valent antibodies used in any of the methods or compositions of the invention can include human antibodies generated by a transgenic non-human animal, such as a mouse, capable of producing human antibodies, as described by, e.g., U.S. Pat. Nos. 5,939,598; 5,877,397; 5,874,299; 5,814,318. In one aspect, the bi-valent antibody comprises an isolated antibody, a synthetic antibody or a recombinantly generated antibody.

In one aspect, the bi-valent antibodies used in the methods and compositions of the invention are made by a process comprising mixing the modified antigen with the bi-valent antibody immediately before administration. In alternative aspects, the compositions (e.g., pharmaceuticals) used in the methods, or the compositions of the invention, are made by a process comprising mixing a modified antigen with an antibody (e.g., a bi-valent or multi-valent) antibody between about 1 minute and two hours before administration, or, mixing modified antigen with the bi-valent antibody between about 10 minutes and one hour before administration, or, mixing modified antigen with the bi-valent antibody between about 30 minutes and one hour before administration.

In one aspect, the compositions (e.g., pharmaceuticals) used in the methods, or the compositions of the invention, are made by a process comprising freeze-drying or lyophilizing the modified antigen and the antigen-specific bi-valent antibody. The freeze-dried mixture can be reconstituted in a formulation for administration at the time of administration. The freeze-dried mixture can be stored at a temperature of between about −20° C. and 4° C. The freeze-dried mixture can be reconstituted in an aqueous formulation, such as sterile distilled water or buffered saline, e.g., PBS, Ringer's and the like.

In one aspect of the compositions (e.g., pharmaceuticals) used in the methods, or the compositions of the invention the antigen comprises a purified or isolated antigen, or, the antigen comprises a recombinant or synthetic polypeptide, or the antigen comprises a soluble antigen or a particulate antigen, or, the autoantigen comprises a small molecular weight antigen, such as an MW of between about 0.1 to 10 kd or about 0.5 to 5 kd, or, the autoantigen comprises a large molecular weight antigen, e.g., an MW of between about 5 to 50 kd or about 10 to 25 kd.

In one aspect of the compositions (e.g., pharmaceuticals) used in the methods, or the compositions of the invention the antigen comprises a cancer-specific antigen or an antigen specific for a hyperplastic cell or tissue. The antigen can comprise a foreign antigen, e.g., a bacterial antigen, a viral antigen, a fungal antigen, a yeast antigen or a protozoan antigen. The antigen can comprise a subcellular fraction, a cell, a tissue or an organ. The cell or the tissue can comprise a subcellular fraction, a cell or tissue homogenate or a cell, tissue or organ extract. The cancer can be melanoma, prostate cancer, thyroid cancer, pancreatic cancer, liver cancer, breast cancer, lung cancer or stomach cancer. The foreign antigen can comprise an antigen from a pathogen or infectious disease agent. The antigen from a pathogen or infectious disease agent can comprise a bacterial antigen, a viral antigen or an antigen from a protozoan, e.g., a Staphylococcus, Streptococcus, E. coli, flu virus, hepatitis A, B or C, or malaria.

In one aspect of the compositions (e.g., pharmaceuticals) used in the methods, or the compositions of the invention, there is about 10%, 20%, 25%, 30%, 35%, 40%, 45%, 50%, 55%, 60%, 65%, 70%, 75%, 80%, 85%, 90%, 95%, 100%, 125%, 150%, 175% or 200% more modified antigen present on a molar basis in the composition than bi-valent antibody. In one aspect, the composition comprises between about 0.1 mg to 10 mg of antigen and an appropriate amount of bi-valent antibody to keep the antigen in molar excess to the bi-valent antibody, or, the composition comprises between about 0.1 mg to 1.0 mg of antigen and an appropriate amount of bi-valent antibody to keep the antigen in molar excess to the bi-valent antibody, or, the composition comprises between about 0.2, 0.3, 0.4, 0.5, 0.6, 0.7, 0.8 or 0.9 mg of antigen and an appropriate amount of bi-valent antibody to keep the antigen in molar excess to the bi-valent antibody.

In one aspect, the composition (e.g., pharmaceutical) is administered parenterally, orally, intranasally or by an ocular route. The composition can be administered once a day, twice a day, or three or more times a day. The composition can be administered about once to twice a week. The composition can be administered initially twice a week for about three weeks, then weekly for about five months, then monthly. In one aspect, once the immune system is tuned to respond to the modified antigen appropriately by the injected complexes of the invention, then injection of modified antigen alone can also maintain the specific immune response (though at a lower immune response level). In one aspect, in order to keep a high level of specific circulating IgG antibodies, the cells of the immune system are stimulated more often than usual by continuous injections of the appropriate complexes of the invention at a slight antigen excess. For example, when cancer cells are eliminated or decrease in number, or solid tumors regress or metastatic cancer sites disappear or diminish, and/or infectious are terminated, or an autoimmune disease or state is ameliorated, a less frequent administration of the appropriate complex of the invention and/or lower dosage of the appropriate complex of the invention can be administered. Successful amelioration of a cancer can be determined by routine procedures, e.g., laboratory tests including biopsy, special serum analysis, imaging procedures (e.g., X-ray, sonogram, MRI) and the like. Successful amelioration of a pathogen or an infectious disease can be determined by routine procedures, e.g., presence of signs, symptoms, laboratory findings and the like.

In one aspect, the compositions (e.g., pharmaceuticals) used in the methods, or the compositions of the invention, comprise a sterile aqueous formulation, such as sterile distilled water or buffered saline, e.g., PBS, Ringer's and the like.

In one aspect, the compositions (e.g., pharmaceuticals) used in the methods, or the compositions of the invention, comprise an adjuvant. In one aspect, the compositions are administered with an adjuvant. The adjuvant can comprise alum or a Freund's adjuvant.

In one aspect, the compositions (e.g., pharmaceuticals) used in the methods, or the compositions of the invention, are modified antigens, e.g., modified antigens from pathogens, disease sources and the like. In one aspect, modified antigens used in the methods or compositions of the invention are different enough from a “tolerated” or “natural” protein to be recognized as foreign, yet similar enough so that it could cross-react with the tolerated protein. While the invention is not limited by any particular mechanism of action, in order for an administered (e.g., injected) antigen (e.g., protein) to provoke an immune response, and thereby terminate an unresponsive state (tolerance), the antigen must be different enough from the “tolerated” protein to be recognized as foreign. In one aspect, the antigen is also similar enough so that it can cross-react with the tolerated protein.

The antigen can be modified by a hapten. Antigen can be haptenized in vitro by various small MW substances to obtain hapten-protein conjugates, such as arsanil-protein, sulfanil-protein, arsanil-sulfanil protein, and the like. In one aspect, an immune-complex is made such that the antigen is at slight molar excess with a specific high titered bi-valent antibody (e.g., IgG, IgA, fused single chain Abs or CDRs) which is directed against it.

The hapten-modified antigen can comprise a hapten-protein conjugate. The hapten-protein conjugate can comprise an arsanil-protein conjugate, a sulfanil-protein conjugate or an arsanil-sulfanil protein conjugate.

The details of one or more aspects of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 graphically illustrates proteinuria in experimental and control animals, as described in detail in Example 1, below.

Like reference symbols in the various drawings indicate like elements.

### DETAILED DESCRIPTION

The invention provides novel methods for manipulating the immune system in a mammal. In one aspect, the invention provides compositions and methods for increasing the levels of an autoantigen-specific IgM antibody in a mammal. Increasing the levels of an autoantigen-specific IgM antibody in a mammal decreases the levels of the autoantigen, including decreasing the soluble, or circulating forms of the autoantigen. Using these autoantigen-specific IgM antibodies, the invention provides compositions and methods for ameliorating, including preventing or treating, an autoimmune disease. In one aspect, the compositions and methods of the invention can be used to ameliorate, including prevent or treat, an allograft rejection, e.g., a tissue, organ or cell (e.g., bone marrow) transplant rejection.

In one aspect, the invention provides compositions and methods for increasing the levels of an antigen-specific IgG antibody in a mammal. Increasing the levels of an antigen-specific IgG antibody in a mammal decreases the levels of the autoantigen, including decreasing the soluble, or circulating forms of the antigen in the mammal. Using these antigen-specific IgG antibodies, the invention provides compositions and methods for ameliorating a disease or condition in a mammal, e.g., a cancer or a foreign antigen, such as a pathogen. The compositions and methods can be used to treat or prevent the disease or condition.

While the invention is not limited by any particular mechanism of action, the methods of the invention are, in part, based on the novel finding that when intracytoplasmic antigens are liberated (e.g., following cell damage), then an immediate production of specific IgM antibodies will occur and play a physiological roll in the clearance of cell debris. Mammals, including humans, are not tolerant to intracytoplasmic particulate antigens. This arm of the autoimmune system is physiologic and therefore beneficial to the individual by providing a most efficient clearance mechanism to remove unwanted, damaged cellular components subsequent to cell death, as a result of injury, infection, trauma, hypoxia or following termination of the life span of a cell.

### General Techniques

The invention provides compositions comprising isolated, recombinant or synthetic autoantigens, antibodies and antigens. The nucleic acids used to practice this invention, e.g., genomic DNA, vectors, viruses or hybrids thereof, may be isolated from a variety of sources, genetically engineered, amplified, and/or expressed/generated recombinantly. Recombinant polypeptides (e.g., autoantigens, antibodies, antigens) generated from these nucleic acids can be individually isolated or cloned and tested for a desired activity. Any recombinant expression system can be used, including bacterial, mammalian, yeast, insect or plant cell expression systems.

Alternatively, nucleic acids and polypeptides used to practice the invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS 1N MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

The invention provides fusion proteins comprising autoantigens, antibodies, antigens used to practice the invention, and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to make a modified antigen, to more readily isolate a recombinantly syntheized peptide (e.g., antigen), to identify and isolate antibodies and antibody-expressing B cells, and the like.

Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein.

Transgenic non-human animals can be used to generate a nucleic acid or a polypeptide (e.g., autoantigens, antibodies, antigens) to practice the invention. The transgenic non-human animals can be, e.g., goats, rabbits, sheep, pigs, cows, rats and mice. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows.

Transgenic plants and plant cells can be used to generate a nucleic acid or a polypeptide (e.g., autoantigens, antibodies, antigens) to practice the invention. Transgenic plants to be used for producing large amounts of the polypeptides (e.g., autoantigens, antibodies, antigens). For example, see Palmgren (1997) Trends Genet. 13:348; Chong (1997) Transgenic Res. 6:289-296 (producing human milk protein beta-casein in transgenic potato plants using an auxin-inducible, bidirectional mannopine synthase (mas1′,2′) promoter with Agrobacterium tumefaciens-mediated leaf disc transformation methods). Using known procedures, one of skill can screen for plants expressing autoantigens, antibodies, antigens of the invention by detecting the increase or decrease of transgene mRNA or protein in transgenic plants. Means for detecting and quantitation of mRNAs or proteins are well known in the art.

Polypeptides and peptides (e.g., autoantigens, antibodies, antigens) used to practice the invention can be isolated from natural sources, be synthetic, or be recombinantly generated polypeptides. Peptides and proteins can be recombinantly expressed in vitro or in vivo. The peptides and polypeptides used to practice the invention can be made and isolated using any method known in the art. Polypeptide and peptides used to practice the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The peptides and polypeptides used to practice the invention can also be glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

Peptides and polypeptides used to practice the invention include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. Polypeptide mimetic compositions used to practice the invention can contain any combination of non-natural structural components. In alternative aspects, mimetic compositions used to practice the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluorophenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, isopentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, in one aspect under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

Polypeptides used to practice the invention can be altered by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

Solid-phase chemical peptide synthesis methods can also be used to synthesize the polypeptide or fragments used to practice the invention, see, e.g., Merrifield (1963) Am. Chem. Soc. 85:2149-2154; Stewart, J. M. and Young, J. D., Solid Phase Peptide Synthesis, 2nd Ed., Pierce Chemical Co., Rockford, Ill., pp. 11-12; commercially available laboratory peptide design and synthesis kits, e.g., Cambridge Research Biochemicals. Such commercially available laboratory kits have generally utilized the teachings of H. M. Geysen et al, Proc. Natl. Acad. Sci., USA, 81:3998 (1984) and provide for synthesizing peptides upon the tips of a multitude of “rods” or “pins” all of which are connected to a single plate.

### Antibodies and Antibody-Based Screening Methods

The invention provides methods and compositions using antibodies, including bi-valent and multivalent antibodies that specifically bind to cancer or pathogenic antigens, or autoantigens, respectively. Antibodies used to practice the invention can be isolated, synthetic or recombinant antibodies.

The antibodies also can be used in immunoprecipitation, staining, immunoaffinity columns, and the like. If desired, nucleic acid sequences encoding for specific antigens can be generated by immunization followed by isolation of polypeptide or nucleic acid, amplification or cloning and expression of polypeptides of the invention. Alternatively, these methods can be used to modify the structure of an antibody, e.g., an antibody's affinity to an antigen (e.g., autoantigen, pathogenic antigen, cancer antigen) can be increased or decreased.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

Antibodies may be used in immunoaffinity chromatography procedures to isolate or purify polypeptides to be used to practice the invention, or to determine whether the polypeptide is present in a biological sample. In immunoaffinity procedures, the antibody is attached to a solid support, such as a bead or other column matrix. The protein preparation is placed in contact with the antibody under conditions in which the antibody specifically binds to a desired polypeptide (e.g., antigen, another antibody). After a wash to remove non-specifically bound proteins, the specifically bound polypeptides are eluted.

The ability of proteins (e.g., antigens) in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays and Western Blots.

For preparation of monoclonal antibodies, any technique which provides antibodies produced by continuous cell line cultures can be used. Examples include the hybridoma technique (Kohler and Milstein, Nature, 256:495-497, 1975), the trioma technique, the human B-cell hybridoma technique (Kozbor et al., Immunology Today 4:72, 1983) and the EBV-hybridoma technique (Cole, et al., 1985, in Monoclonal Antibodies and Cancer Therapy, Alan R. Liss, Inc., pp. 77-96).

Techniques described for the production of single chain antibodies (see, e.g., U.S. Pat. No. 4,946,778) can be adapted to produce single chain antibodies for use in the methods and compositions of the invention.

As discussed above, transgenic mice may be used to express human or humanized antibodies for use in the methods and compositions of the invention.

### Kits

The invention provides kits comprising the compositions, e.g., immune complexes and/or pharmaceuticals of the invention. The kits also can contain instructional material teaching the methodologies and industrial uses of the invention, as described herein.

### Autoantigens and Autoimmune Diseases

The invention provides methods and compositions for increasing the levels of an autoantigen-specific IgM antibody in a mammal, decreasing the levels of an autoantigen, and ameliorating an autoimmune disease. Autoantigens used in the compositions and methods of the invention and their corresponding disease targeted by the methods of the invention include, for example: myelin basic protein (MBP) and multiple sclerosis (MS); oligodendrocyte glycoprotein, myelin basic protein (MBP) and experimental autoimmune encephalitis (EAE); acetylcholine receptor and myasthenia gravis; insulin, 1A-2 antigen, glutamic acid decarboxylase (GAD) and type I diabetes; thyroglobulin and autoimmune thyroiditis; collagen type IV α3-chain and Goodpasture syndrome; fibrillarin and scleroderma.

In one aspect, the compositions and methods of the invention use modified or unmodified cryptic autoantigens, including cryptic autoantigens, and/or dominant autoantigens (autoantigens which are exposed to immunological cells, e.g., on surfaces of blood cells in the circulation, within tissues etc.). Modified autoantigens after administration are recognized as foreign and initiate an immune response, e.g., a pathogenic immune response, which can comprise humoral (IgG) and/or cell mediated responses.

Insulin-Dependent Diabetes Mellitus (IDDM)

The invention provides methods and compositions for ameliorating autoimmune insulin-dependent diabetes mellitus (IDDM), an autoimmune reaction to insulin-secreting beta-cells of the pancreas. See, e.g., U.S. Pat. No. 6,214,985. Methods and compositions of the invention use antigens associated with IDDM, including glutamate acid decarboxylase isoforms, insulin, carboxypeptidase H, ICA 516 and 64 kD integral membrane proteins, hsp65, several secretory granule protein (e.g., insulin-secretory granule antigens), mouse insulin-secretory granule antigen (imogen 38).

Insulin-dependent diabetes mellitus (IDDM) is an autoimmune disease that results from the destruction of the insulin-secreting beta-cells of the pancreas. Patients with IDDM have insulitis, a lymphocytic infiltration of the islets of Langerhans, islet-specific Th1 lymphocytes, and antibodies directed against components of the islet cells.

Methods and compositions of the invention for ameliorating IDDM can be used, and tested, in IDDM animal models. IDDM in animal models is T cell mediated and requires the participation of both CD8+, class I MHC restricted and CD4+, class II MHC restricted T cells. There is a demonstrated association between MHC class II DR4 polymorphic alleles and disease susceptibility, indicating that the response is antigen driven.

Methods and compositions of the invention use the several beta-cell proteins that have been identified as antigens in IDDM, including two glutamate acid decarboxylase isoforms, insulin, carboxypeptidase H, ICA 516 and 64 kD integral membrane proteins, hsp65, several secretory granule protein (e.g., insulin-secretory granule antigens), mouse insulin-secretory granule antigen (imogen 38). Some of these antigens have been found in the sera of diabetic and prediabetic individuals. See, e.g., U.S. Pat. Nos. 6,211,352; 6,025,176; 5,792,620.

Autoantibodies reactive with glutamic acid decarboxylase GAD in GABA-ergic neurons are present in the majority of sera from patients with the rare neurological disease Stiff Man Syndrome. Patients positive for GAD autoantibodies have an increased frequency of polyendocrine autoimmunity, e.g., IDDM. During the pre-clinical stage of IDDM and in patients with recent onset clinical IDDM, autoantibodies are frequently detected against an islet cell MW 64,000 protein, or a form of GAD.

Systemic Lupus Erthyematosus (SLE)

The invention provides methods and compositions for ameliorating systemic lupus erthyematosus (SLE). Methods and compositions of the invention use antigens associated with systemic lupus erthyematosus, including nucleolus protein ASE-1, fibronectin, cardiolipin, histone H2A-H2B-DNA, KU-DNA protein kinase, golgin and/or collagen, Ro/SSA, La/SSB, nRNP, Sm, HP-8. See, e.g., U.S. Pat. Nos. 6,177,254; 6,111,088; 5,807,993.

Indirect immunofluorescence analysis using antibodies generated to cloned regions of nucleolus protein ASE-1 indicates that this protein occurs at the fibrillar centers of the nucleolus in the putative sites of rDNA transcription. During cell division ASE-1 localizes to the nucleolus organizer regions of the chromosomes, where it is closely associated with RNA polymerase. As an autoantigenic nucleolar protein, ASE-1 has been found to be a reliable serum marker for systemic lupus erthyematosus (SLE). This finding makes ASE-1 useful in the clinical detection and characterization of the disease. To identify the presence of SLE in an individual patient, a serum samples can be taken and screened against the cloned ASE-1 protein to identify sera with anti-ASE-1 autoantibodies. This screening can be done using an ELISA assay, western blot techniques, or by binding the antigen to microspheres and identifying reactive sera by flow cytometry.

The production of circulating autoantibodies to ribonucleoprotein complexes (RNPs) is a unifying characteristic of some of the rheumatic autoimmune diseases. The most common antigens in SLE and closely related disorders include: Ro/SSA, La/SSB, nRNP and Sm. Initially, these antibodies were found using double immunodiffusion, but more recently sensitive solid phase assays have been developed to quantitate the autoantibodies. The Ro/SSA RNA-protein particle has been found to be a constituent of all human cells evaluated to date.

Another antigen used in the methods and compositions of the invention to treat SLE is HP-8. The HP-8 transcripts are expressed in brain, heart, placenta, lung, skeletal muscle, pancreatic tissues, and kidney:

Endometriosis

The invention provides methods and compositions for ameliorating endometriosis. Methods and compositions of the invention use antigens associated with endometriosis, e.g., Repro-EN-1.0, IB1. See U.S. Pat. No. 6,525,187.

Endometriosis is a painful disorder that is characterized by the ectopic implantation of functioning endometrial tissue into the abdominal wall and the outer surface of various organs including, most commonly, the lower bowel, ovaries and fallopian tubes. P. Vigano et al. (1991) Fertility and Sterility 56:894. Endometriosis has an autoimmune component. IgG and IgA auto-antibodies that react with multiple endometrial antigens have been documented in patients with endometriosis. Studies have shown that circulating IgG antibodies that bind multiple endometrial proteins can be detected in women with endometriosis to varying degrees. Thirty-five percent to 74% of patients have sera reactive with endometrial proteins, see, e.g., Odukoya (1996) Acta Obstet. Gynecol. Scand. 75:927-931; Kim (1995) Am. J. Reprod. Immunol. 34:80-87; Odukoya (1995) Hum. Reprod. 10:1214-1219.

Repro-EN-1.0, and its alternately spliced variant IB1 are used in the compositions and methods of the invention. Subjects diagnosed with endometriosis have been found to have antibodies that specifically bind to Repro-EN-1.0 polypeptide and/or a IB1 polypeptide. These antibodies represent a highly sensitive and specific diagnostic marker for endometriosis. Recombinant Repro-EN-1.0 protein and recombinant IB1 protein are useful to detect such antibodies in immunoassays.

Acquired Hypoparathyroidism (AH)

The invention provides methods and compositions for ameliorating acquired hypoparathyroidism (AH). Methods and compositions of the invention use antigens associated with acquired hypoparathyroidism (AH), including calcium sensing receptor (CA-SR). See, e.g., U.S. Pat. No. 6,066,475.

Acquired hypoparathyroidism (AH) patients react to cytosolic antigens of 70 kDa, and 80 kDa, and to a membrane associated antigen of 120-140 kDa, calcium sensing receptor (CA-SR). The findings of parathyroid specific autoantibodies in many patients with AH document the autoimmune nature of this disease, while the localization of the reactive epitope of the CA-SR to its external domain suggests that activation of the receptor could induce inhibition of PTH secretion in the disease.

Multiple Sclerosis (MS)

The invention provides methods and compositions for ameliorating multiple sclerosis (MS). Methods and compositions of the invention use antigens associated with MS, including myelin basic protein (MBP), transaldolase. See, e.g., U.S. Pat. Nos. 6,018,021; 5,879,909.

Primary Biliary Cirrhosis (PBC)

The invention provides methods and compositions for ameliorating primary biliary cirrhosis (PBC). Methods and compositions of the invention use antigens associated with primary biliary cirrhosis (PBC), including mitochondrial antigens. See, e.g., U.S. Pat. No. 5,891,436.

Primary biliary cirrhosis (PBC) is a chronic disease characterized by progressive inflammatory obliteration of the intrahepatic bile ducts. The disease is marked by an autoantibody response to mitochondria, originally identified using immunofluorescence. Specific proteins have been recognized as targets of the anti-mitochondrial antibodies (AMA) of PBC. In particular, serum antibodies to a 70 kilodalton (kd) protein have been found in greater than 95% of patients with PBC but not in patients with other autoimmune liver diseases.

Rheumatoid Arthritis (RA)

The invention provides methods and compositions for ameliorating rheumatoid arthritis. Methods and compositions of the invention use antigens associated with rheumatoid arthritis (RA), including Type II collagen, osteopontin, proteoglycans, fibronectin, glucose 6-phosphate isomerase, keratin, golgin, HC gp-39. See, e.g., U.S. Pat. Nos. 5,869,093; 5,843,449.

The invention provides methods and compositions for use in studies involving adjuvant arthritis (AA), which is an experimental model of inflammatory joint disease, e.g., a model of rheumatoid arthritis. Adjuvant arthritis is induced by intradermal injection of a suspension of Mycobacterium tuberculosis (MT) in oil. Between 10 and 15 days following injection, animals develop a severe, progressive arthritis.

Because of its resemblance to human rheumatoid arthritis in both clinical and histopathological features, AA has been used as a model to investigate mechanisms of immune mediated joint disease and to investigate methods for the treatment of an organ specific autoimmune disease, and can be used to determine formulations, dosages, etc. in the methods and compositions of the invention.

Human cartilage glycoprotein 39 (HC gp-39) is a target autoantigen in RA patients which activates specific T cells, thus causing or mediating the inflammatory process. HC gp-39 derived peptides were predominantly recognized by autoreactive T cells from RA patients but rarely by T cells from healthy donors, thus indicating that HC gp-39 is an autoantigen in RA.

Autoimmune Infertility

The invention provides methods and compositions for ameliorating autoimmune infertility. Methods and compositions of the invention use antigens associated with autoimmune infertility, including mammalian Sp 17 protein. See, e.g., U.S. Pat. No. 5,820,861.

Autoimmune Addison's Disease

The invention provides methods and compositions for ameliorating autoimmune Addison's disease. Methods and compositions of the invention use antigens associated with autoimmune Addison's disease, including adrenal autoantibodies. See, e.g., U.S. Pat. No. 5,705,400.

An epitope for an adrenal autoantibody has an observed molecular weight of from about 50,000 to about 60,000 and is obtainable by: homogenizing adrenal glands, subjecting the homogenate to differential centrifugation to obtain a microsome fraction, suspending the microsome fraction in a phosphate buffer, centrifuging the suspension in the presence of sodium cholate to form a supernatant, adding polyethylene glycol and further sodium cholate to the supernatant and mixing the supernatant, centrifuging the thus mixed supernatant to form a pellet, resuspending the pellet in aqueous sodium cholate to form a suspension, dialyzing the suspension against aqueous sodium cholate to form a solubilized microsome preparation, and purifying the solubilized microsome preparation by column chromatography to obtain a column fraction containing the protein. The protein can be obtained from human adrenal glands.

### Formulation and Administration of Pharmaceuticals

In one aspect, the invention provides pharmaceutical compositions comprising an unmodified autoantigen and an antigen-specific multi-valent antibody. In one aspect, the invention provides pharmaceutical compositions comprising a modified antigen and an antigen-specific bi-valent antibody. In one aspect, the pharmaceutical compositions are formulations that comprise a pharmacologically effective amount of these antibodies and antigens. In one aspect, a pharmacologically effective amount of a pharmaceutical composition of the invention is an amount sufficient to ameliorate an autoimmune disease (when compositions comprising an unmodified autoantigen and an antigen-specific multi-valent antibody are administered) or ameliorate a disease or condition associated with a foreign antigen or a pathogen-associated antigen, such as a cancer antigen, a bacterial or viral antigen, and the like (when compositions comprising a modified antigen and an antigen-specific bi-valent antibody is administered). In alternative aspects, by ameliorating an autoimmune disease or a disease or condition associated with a foreign antigen or a pathogen-associated antigen, the methods and compositions of the invention can treat, lessen the severity of, slow or prevent the onset of, and/or slow the progress of the autoimmune disease or disease or condition associated with a foreign antigen or a pathogen-associated antigen.

The pharmaceuticals of the invention can be administered by any means in any appropriate formulation. Routine means to determine drug regimens and formulations to practice the methods of the invention are well described in the patent and scientific literature. For example, details on techniques for formulation, dosages, administration and the like are described in, e.g., the latest edition of Remington's Pharmaceutical Sciences, Maack Publishing Co, Easton Pa.

The formulations of the invention can include pharmaceutically acceptable carriers that can contain a physiologically acceptable compound that act, e.g., to stabilize the composition or to increase or decrease the absorption of the pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipients or other stabilizers and/or buffers. Detergents can also used to stabilize the composition or to increase or decrease the absorption of the pharmaceutical composition. Other physiologically acceptable compounds include wetting agents, emulsifying agents, dispersing agents or preservatives that are particularly useful for preventing the growth or action of microorganisms. Various preservatives are well known, e.g., ascorbic acid. One skilled in the art would appreciate that the choice of a pharmaceutically acceptable carrier, including a physiologically acceptable compound depends, e.g., on the route of administration and on the particular physio-chemical characteristics of any co-administered agent.

In one aspect, the composition for administration comprises a pharmaceutically acceptable carrier, e.g., an aqueous carrier. A variety of carriers can be used, e.g., buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration and imaging modality selected.

The pharmaceutical formulations of the invention can be administered in a variety of unit dosage forms, the general medical condition of each patient, the method of administration, and the like. Details on dosages are well described in the scientific and patent literature, see, e.g., the latest edition of Remington's Pharmaceutical Sciences. The exact amount and concentration of pharmaceutical of the invention and the amount of formulation in a given dose, or the “effective dose” can be routinely determined by, e.g., the clinician (see above discussion of a pharmacologically effective amount of a composition of the invention). The “dosing regimen,” will depend upon a variety of factors, e.g., the general state of the patient's health, age and the like. Using guidelines describing alternative dosaging regimens, e.g., from the use of other imaging contrast agents, the skilled artisan can determine by routine trials optimal effective concentrations of pharmaceutical compositions of the invention. The invention is not limited by any particular dosage range and the pharmaceuticals of the invention can be administered by alternative dosages.

For example, the amount of antigen, whether soluble, particulate, sonicated or partially degraded (which can be expressed as mg/ml in the composition) can be varied according to size and/or weight of the recipient in order to acquire the best possible desired immune response. The amount of antibody, with a known antibody titer against the antigen, can be adjusted in such manner that the Ag:Ab complex will be at a slight antigen excess.

For example in one aspect the composition is administered according to the following vaccination protocol: initially twice a week for three weeks then after weekly for five months, then after monthly (frequency of administration will depend on signs, symptoms and laboratory findings). In order to keep a high level of specific circulating IgM autoantibodies, the cells of the immune system will be stimulated more often than usual by continuous injections of the appropriate Ab:Ag complexes of the invention at a slight Ag excess. When termination of the pathogenic antibody response is achieved a less frequent administration of the Ab:Ag complexes of the invention can be instituted, including Ab:Ag complexes at molar equivalence or at antibody excess. However, in both formulations, especially at Ab excess, the antibody response is depressed.

In one aspect, once the immune system is tuned to respond to the modified antigen by the administered (e.g., injected) Ab:Ag complexes of the invention then administration of the modified antigen alone can also maintain the specific immune response (though at a lower immune response level).

In practicing the methods of the invention, the appropriate dosage can be determined by the skilled clinician. In one aspect, the amount of antigen, whether soluble, particulate, sonicated, or partially degraded, expressed as mg/ml in the pharmaceutical composition, can be varied according to size/weight of the recipient in order to acquire the best possible immune response for a desired period of time. The amount of antibody, with a known antibody titer against the antigen, can be adjusted in such manner that the Ag:Ab complex of the invention will be at a slight antigen excess. The presentation (e.g., method of administration) of the antigen, frequency of antigen administration, antigen dose, the amount of antigen excess in the Ag:Ab complexes of the invention, and the like, will determine the immune response. Generally speaking, a low dose of antigen in the Ag:Ab complexes of the invention will initiate and maintain an elevated immune response in the individual (antibody information transfer) against the antigen present in the Ag:Ab complex by the same class of antibody which is present in the Ag:Ab complex.

The pharmaceutical compositions of the invention can be delivered by any means known in the art systemically (e.g., intravenously), regionally, or locally (e.g., intra- or peri-tumoral or intracystic injection) by, e.g., intraarterial, intratumoral, intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa), intra-tumoral (e.g., transdermal application or local injection). For example, intra-arterial injections can be used to have a “regional effect,” e.g., to focus on a specific organ (e.g., brain, liver, spleen, lungs).

Formulations suitable for oral administration can comprise liquid solutions, such as an effective amount of the compound dissolved in diluents, such as water, saline, or fruit juice; capsules, sachets or tablets, each containing a predetermined amount of the active ingredient, as solid, granules or freeze-dried cells; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Suitable formulations for oral delivery can also be incorporated into synthetic and natural polymeric microspheres, or other means to protect the agents of the present invention from degradation within the gastrointestinal tract. See, for example, Wallace (1993) Science 260:912-915.

The pharmaceutical compositions of the invention can be made into aerosol formulations to be administered via inhalation. These aerosol formulations can be placed into pressurized acceptable propellants, such as dichlorodifluoromethane, propane, nitrogen and the like.

The cyanovirins or conjugates thereof, alone or in combinations with other antiviral compounds or absorption modulators, can be made into suitable formulations for transdermal application and absorption. Transdermal electroporation or iontophoresis also can be used to promote and/or control the systemic delivery of a polypeptide of the invention through the skin, e.g., see Theiss (1991) Meth. Find. Exp. Clin. Pharmacol. 13:353-359.

Formulations suitable for topical administration of a pharmaceutical compositions of the invention can include lozenges comprising the active ingredient in a flavor, usually sucrose and acacia or tragacanth; pastilles comprising the active ingredient in an inert base, such as gelatin and glycerin, or sucrose and acacia; and mouthwashes comprising a pharmaceutical compositions of the invention in a suitable liquid carrier; as well as creams, emulsions, gels and the like.

Formulations suitable for parenteral administration can include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The pharmaceutical formulations of the invention can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Therapeutic compositions can also be administered in a lipid formulation, e.g., complexed with liposomes or in lipid/nucleic acid complexes or encapsulated in liposomes, as in immunoliposomes directed to specific cells. These lipid formulations can be administered topically, systemically, or delivered via aerosol. See, e.g., U.S. Pat. Nos. 6,149,937; 6,146,659; 6,143,716; 6,133,243; 6,110,490; 6,083,530; 6,063,400; 6,013,278; 5,958,378; 5,552,157.

In one aspect, pharmaceutical formulations of the invention can be used with an absorption-enhancing agent. Any absorption-enhancing agent can be, used, e.g., those applied in combination with protein and peptide drugs for oral delivery and for delivery by other routes, see, e.g., van Hoogdalem, Pharmac. Ther. 44, 407-443, 1989; Davis, J. Pharm. Pharmacol. 44(Suppl. 1), 186-190, 1992. Enhancers used in the compositions and methods of the invention include, e.g., (a) chelators, such as EDTA, salicylates, and N-acyl derivatives of collagen, (b) surfactants, such as lauryl sulfate and polyoxyethylene-9-lauryl ether, (c) bile salts, such as glycolate and taurocholate, and derivatives, such as taurodihydrofusidate, (d) fatty acids, such as oleic acid and capric acid, and their derivatives, such as acylcarnitines, monoglycerides and diglycerides, (e) non-surfactants, such as unsaturated cyclic ureas, (f) saponins, (g) cyclodextrins, and (h) phospholipids.

Other approaches to enhancing oral delivery of protein and peptide drugs (e.g., the antigen:antibody complexes of the invention) are also used the practice the invention, e.g., chemical modifications to enhance stability to gastrointestinal enzymes and/or increased lipophilicity. Alternatively, or in addition, the pharmaceutical formulations of the invention can be administered in combination with other drugs or substances, which directly inhibit proteases and/or other potential sources of enzymatic degradation of proteins and peptides. Another alternative approach to prevent or delay gastrointestinal absorption of cyanovirin is to incorporate it into a delivery system that is designed to protect the protein or peptide in the pharmaceutical formulations of the invention from contact with the proteolytic enzymes in the intestinal lumen and to release the intact protein or peptide only upon reaching an area favorable for its absorption. In one aspect, a biodegradable microcapsules or microspheres is used with pharmaceutical formulations of the invention, both to protect them from degradation, as well as to effect a prolonged release of active drug, see, e.g., Deasy, in Microencapsulation and Related Processes, Swarbrick, ed., Marcell Dekker, Inc.: New York, 1984, pp. 1-60, 88-89, 208-211. Microcapsules also can provide a useful way to effect a prolonged delivery of pharmaceutical formulations of the invention after injection, see, e.g., Maulding, J. Controlled Release 6, 167-176, 1987.

### EXAMPLES

### Example 1

### Downregulation of Pathogenic Autoantibody Responses

The following example demonstrates that the compositions and methods of the invention are effective in the downregulation of pathogenic autoantibody responses in mammals. The invention describes for the first time an antigen-specific downregulation of pathogenic autoantibody mediated disease process. The antigen-specific downregulation is demonstrated in a model for autoimmune kidney disease, an experimental autoimmune kidney disease of rats called Slowly Progressive Heymann Nephritis (SPUN), see Example 2, below. This autoimmune disease is initiated and maintained by pathogenic autoantibodies, causing immune-complex glomerulonephritis resulting in proteinuria.

Pathogenic autoantibody responses were downregulated in SPHN rats by injections of exemplary immune-complexes of the invention containing the native nephritogenic antigen and specific IgM autoantibodies, in antigen excess. The injected immune complexes raised the level of circulating non-pathogenic IgM autoantibodies, which in turn, by removing the injected altered nephritogenic and liberated autoantigen (from the renal tubules), greatly reduced the production of pathogenic autoantibodies and continuous build up of immune deposits in the glomeruli. Animals treated early responded better than rats treated late into their diseases, but considering improvements in proteinuria, kidney lesion reduction and pathogenic autoantibody response, they all did well. At the end of the experiment at 29 weeks 80% of rats had insignificantly low levels of circulating IgG autoantibodies indicating cessation of pathogenic autoantibody production with corresponding termination of the disease process. On the other hand untreated rats at the end of the experiment still had high levels of circulating pathogenic autoantibodies indicating continued disease progression.

As noted above, Heymann nephritis (HN) is an art-recognized model for autoimmune kidney disease. It is a true autoimmune kidney disease of rats first described by Heymann and colleagues, see, e.g., Adorini (1993) Immunol. Today 14:285-289. It is one of the best studied experimental autoimmune kidney disease models. The disease is initiated and maintained by pathogenic autoantibodies see, e.g., Glassock (1968) J. Exp. Med. 127:573-588; Mendrick (1980) Kidney Int. 18:328-343. Following IP or intra-footpad injections of homologous chemically modified or unmodified renal tubular antigens, most often incorporated into FCA, classical HN develops characterized by immune-complex glomerulonephritis and proteinuria.

Autologous and heterologous renal tubular antigens can be also used with adjuvants to produce the kidney disease. The nephritogenic antigen was purified and characterized by several investigators and the preparations were designated as gp 330 and gp 600 respectively, see, e.g., Kerjaschki (1982) Proc. Natl. Acad. Sci. U.S.A 79:5557-5581. Other smaller MW nephritogenic antigens have also been identified. When injected in a suitable form all the purified antigens were able to induce the disease in susceptible breeds of rats, see, e.g., Kerjaschki (1983) J. Exp. Med. 157:667-686. Immunopathological events, leading to morphological changes in the kidney are well described by histological, fluorescent antibody and electron-microscopical studies; and functional changes characterized by circulating pathogenic autoantibodies and proteinuria are well documented.

The invention also provides a novel, slowly progressive HN(SPHN) kidney disease model: Animals with SPHN started to develop proteinuria from 17 weeks after the induction of the disease, and kidney lesions were also less severe at the early stages. This new experimental autoimmune kidney disease model was used to demonstrate to efficacy of the compositions and methods of the invention to remove specifically circulating nephritogenic antigens and thereby prevent them from stimulating pathogenic autoantibody producing immune cells. Thus, the compositions and methods of the invention also help prevent immune complex deposition in the glomeruli. The present study investigated the effects of intervention using compositions and methods of the invention in two groups of animals. In one group of rats intervention started before the experiment begun and continued throughout the experiment and in the other group from 14 weeks after the disease was established. Untreated normal rats and animals with the kidney disease served as controls.

Animals: Two month old male Sprague Dawley rats were used in the experiment. Animals obtained from the local breeding colony were numbered by an ear identification system and randomly assigned to the metabolic control and test groups. The invasive experimental procedures were carried out on Isoflurane anaesthetized rats. At the end of the experiment, 29 weeks after the induction of the disease, rats were euthanized by IP injections of Euthanyl (MTC pharmaceuticals) (180 mg/kg body weight).

Experimental Design

Group I metabolic controls: 8 rats were included in this group. These animals were not injected or treated. However, weekly proteinuria studies, regular blood collections and kidney samples were obtained from these rats at the same time as for rats in the other groups.

Group II slowly progressive Heymann nephritis (SPHN): 10 rats were subcutaneously injected with sonicated ultracentrifuged (u/c) Azo-rKF3 antigen in Alum and Distemper complex virus vaccine (Olson 30) by a technique described previously (B+C+B+L 29). Rats were injected three times with 0.2 ml of antigen adjuvant mixture. On day 0 the mixture contained 160 μg and on days 16 and 33, 80 μg antigen. Additional six subcutaneous (SC) injections of 160 μg aqueous sonicated u/c Azo-rKF3 antigen were administered on days 26, 43, 65, 72, 79, and 86. The dorsal surface between the shoulder blades was used for all the SC injections.

Group III Pre- and Post-treated rats with SPHN: 10 rats (27 days before the induction of SPHN by the same protocol used in group II animals) were pre-treated with 0.2 ml IP injections of antigen or a combination as follows: On day −27 with 500 μg aqueous rKF3 antigen/rat. On days −22, −20, and −15 with 100 μg aqueous rKF3 antigen/rat. On day −12 with 0.2 ml immune complexes with specific IgM antibodies (MICs) containing 60 μg sonicated u/c rKF3 antigen and 150 μg rarKF3 IgM/rat and on days −8, −5, and −1 with MICs containing 30 μg sonicated u/c rKF3 antigen and 75 μg rarKF3IgM/rat. After the induction of the SPHN rats were post-treated with 0.2 ml MICs twice weekly containing 30 μg sonicated u/c rKF3 antigen and 75 μg rarKF3IgM/rat for the first four weeks and then after the same dose of MICs were administered weekly till the end of the experiment.

Group IV—Post-treated rats with SPHN: SPHN was induced in 10 rats by the same protocol as described for group II rats. From 14 weeks after the induction of the disease rats were treated weekly with 0.2 ml IP injections of MICs containing 30 μg sonicated u/c rKF3 antigen and 75 μg rarKF3IgM/rat.

Preparation of rat kidney tubular fraction 3 antigen (rKF3): Normal rat kidneys were obtained from euthanized adult Sprague Dawley rats following bleeding and washing out of their blood vessels with cold physiological saline. Kidney samples were collected and washed several times with 0.25 mol/L buffered sucrose solution pH 7.4 and homogenized to make a fine suspension. Rat kidney fraction 3 was obtained by differential centrifugation at 4° C. by techniques described herein.

Preparation of a sonicated ultracentrifuged (u/c) Azo-rKF3: A two step procedure was employed. Protein concentration of the previously prepared rKF3 fraction was determined and adjusted to 10 mg/ml before being sonicated and ultracentrifuged. The protein content of the ultracentrifuged supernatant, designated as the sonicated u/c rKF3 preparation, was adjusted to 4 mg/ml, see Example 2. The chemical coupling of the sonicated u/c rKF3 preparation took place in a 0.1 mol/L buffered borax solution at pH 8.2 with diazonium salt. Following exhaustive dialysis of the azo-protein preparation (to get rid of uncoupled diazonium salt), its protein content was adjusted to 4 mg/ml (B+L, 29).

Preparation of rat anti-rKF3 IgM: The low level of circulating naturally occurring IgM autoantibodies directed against the renal tubular BB regions can be boosted. See, e.g., Weir (1966) Clin. Exp. Immunol. 1:433-442. Adult Wistar rats were injected by weekly IP administration of 0.2 ml 50 μg rKF3 antigen in PBS for 4 weeks. Four days after the last injection rats were bled for sera and individual serum samples were tested in an indirect fluorescent antibody test on normal rat kidney sections. Sera of rats with high IgM antibody activity against tubular bb related antigens between 1:70-1:180 titers were pooled and bottled in aliquots needed for MIC preparations and stored at −35° C. until use. To obtain additional rarKF3IgM antibodies, rats were re-stimulated as required for the experiment.

Preparation of sonicated u/c rKF3×rarKF3IgM immune complexes designated as MICs: The following reagents were needed for the preparation of MICs. Sonicated u/c rKF3 antigen: 5 mg/ml and rarKF3 IgM antibody with approximately 1:120 activity against tubular BB related antigens. The IgM concentration of our preparation was considered to be about 2 mg/ml. Fresh preparations of MICs were made prior to injections. Each rat received 30 μg sonicated u/c rKF3 antigen and 75 mg rarKF3 IgM in the IC unless otherwise stated. For example, to make MICs for IP injections of 10 rats the following procedure was employed: 300 μg sonicated u/c rKF3 antigen 750 μg rarKF3 IgM antibody was given and its volume adjusted to 2 ml with PBS. The mixture was incubated and rotated at room temperature (RT) for 30 minutes prior to injection. The MIC preparation was considered to be at slight antigen excess.

Light microscopy: Cortical renal tissues were fixed in 10% neutral buffered formalin and embedded in paraffin. Three μm thick sections were stained with haematoxylin and eosin, the periodic acid-Schiff reaction and by the methenamine silver stain, as described by Barabas (1969) Clin. Exp. Immunol. 5:419-427.

Electron microscopy: One mm blocks of renal cortex were fixed in buffered glutaraldehyde, post fixed in Caulfield's osmium tetroxide and embedded in Peon. Appropriately stained thin sections were examined with a Hitachi 11600 electron microscope, as described by Barabas (1969) Clin. Exp. Immunol. 5:419-427.

Urinary protein estimation: Twenty four hours of urine samples were collected from individual rats in metabolic cages 8 weeks before the start of the experiment to obtain representative baseline values. Weekly collection of urine continued till the end of the experiment at 29 weeks. Urinary protein content was determined on 0.5 ml samples of urine by a biurette method using a Spectromic GENESIS 5™ spectrophotometer at 540 nm.

Immunofluorescent Studies

Direct fluorescent antibody test: Three μ thick fresh kidney sections of individual rats at 8 weeks and at the end of the experiment were cut on a Microm HM500M cryostat and fixed in ether:alcohol (50:50). Washed kidney sections were stained for rat IgG with a suitable dilution of Alexa Fluor® 488 goat anti-rat IgG (H+L) (Molecular Probe) and for rat IgM with a suitable dilution of Alexa Fluor® goat anti-rat IgM ((μ)chain) (Molecular Probe). At the end of the experiment kidney sections were also stained for C5b-9 with a monoclonal mouse anti-rat C5b-9 IgG antibody and counter-stained with a suitable dilution of Alexa Fluor® 488 highly absorbed goat anti-mouse IgG (H+L) (Molecular Probe).

Indirect fluorescent antibody test: Dilutions of sera from all the rats obtained at 0, 2, 8, 16, 22, and 29 weeks were tested for rat IgG and IgM antibody activity against rat kidney tubular components on frozen sections of normal rat kidneys. Antibody titers in both IgG and IgM antibodies were recorded and expressed as reciprocals of the last dilution giving a positive result and tabulated and G/M ratios were also calculated. Immunofluorescent antibody stained sections were viewed with a Axioscop Zeiss microscope and digital pictures were taken and stored in a Micron computer.

Grading of glomerular lesions resulting from the deposition of rat IgG in the glomeruli: The most abundant immunoglobulin, rat IgG, which was responsible for the autoimmune pathology in the kidney, was graded. The intensity of fluorescence and the amount of fluorescent material (the beaded immune-complexes) in the glomeruli were graded on a 0-4+ scale according to descriptions described below. Presence of rat IgG in the tubular basement membrane (TBM), brush-border regions of the proximal renal tubules (BB) and Bowman's capsules were also observed and recorded.

The presence of rat IgM was observed in the mesangium and glomerular capillaries also. Beaded mesangial deposits were graded on a 0-4+ scale for fluorescent intensity and for the amount of fluorescent material. Minimal amount of beaded deposition of rat IgM around the glomerular capillaries was also observed and recorded.

Proteinuria: Baseline proteinuria measurements were obtained from all the rats on 8 weekly collections of urine samples before the induction of the kidney disease. Urine was also collected to see if pre-treatment of group III rats with MICs would effect proteinuria. Untreated rats with SPHN had the highest levels of proteinuria, while the pre- and post-treated group III rats had more or less the same levels of proteinuria, throughout the experiment, as group I metabolic controls. Group IV rats post-treated with MICs showed somewhat increased levels of proteinuria. When we compared average daily proteinuria results to control group I rats' urinary protein outputs at the end of the experiment, then SPUN pre- and post-treated rats had 12% higher, post-treated rats 81% higher and untreated rats 230% higher urinary protein losses, showing significant decreases in proteinuria values in the treated rats.

Early treatment of group III rats resulted in very insignificant increases in proteinuria

Histology

| | | |
|---|---|---|
| Group I | 8, 13 | |
| Normal rats: | 2, 3, 6, 8 in I after I el. γG. | (6) |
| SPHN Group II | 9, 10, 17, 25, 47, 50, 65, 81, 83, 87 | (7) |
| SPHN p/p TX (III) Group VI | 21, 40, 41, 43, 44, 56, 61, 68, 76 | (5) |
| SPHN/p TX (IV) Group VII | 3, 7, 12, 16, 20, 45, 51, 66, 83, 89, 92 | (6) |

Light Microscopy: H & E sections revealed no significant changes in the glomeruli of SPHN group II, III, and IV rats but a slight increase in glomerular cellularity was observed in the kidneys of all the SPHN animals. Methenamine silver stained kidney sections of proteinuric group II rats revealed thickened and often vacuolated glomerular capillaries with numerous silver positive projections around their outer circumferences and prominent mesangial areas.

Electron microscopy: Metabolic group I control rats showed no immune deposits in their glomeruli. Group H proteinuric animals revealed the typical HN-kidney lesions. There were small to large electron dense deposits on the epithelial side of the GBM partially or completely surrounded by basement membrane (BM)-like materials. The BM-like projections irregularly thickened the GBM and in relation to the deposits foot-process fusions were observed. The epithelial cell showed patchy osmiophilic areas, especially opposite to the deposits. Pre- and Post-treated Group III rats showed mild forms of HN-kidney lesions. In these animals the GBMs were not thickened and the occasional deposits, sitting on the epithelial side of the GBM were without BM-like projections. Foot-processes were retained in most areas and were fused only in relation to the deposits. In group IV rats, the HN-kidney lesions were somewhat in between group II and III rats' kidney lesions. Some of the deposits were confined on the epithelial side of the GBM in areas where the GBM-like material started to have projections encircling or enclosing deposits. In these areas, epithelial cells were fused and osmiophilic-areas were present in the epithelial cell cytoplasm. In other areas deposits were sitting on the epithelial side of the GBM without apparent changes in the GBM.

Direct fluorescent antibody test results: Table 1, below, shows a number of important observations on the kidney sections of rats in groups II, III and N by the direct fluorescent antibody test results. At week 8, kidney biopsies with different degrees of advanced lesions were observed in the glomeruli of group II, III and IV rats. Moderate beaded glomerular capillary depositions with intense fluorescent staining for rat IgG were observed on the kidney sections of group II and IV rats. In group III rats, minimal deposition of rat IgG with correspondingly less intense fluorescence was observed around the glomerular capillaries. On some of the rat kidney sections tubular basement membranes (TBMs), BBs and Bowman's capsules (BCs) were staining in group II and IV rats only. Metabolic control rats' kidney sections did not stain for rat IgG. Mesangial regions of rat kidney sections stained for rat IgM with a similar fluorescent intensity and grades in all groups of rats including metabolic controls. Treatment or no treatment made no apparent differences during the early stages in mesangial deposits.

At week 29, at the end of the experiment, glomerular depositions with more intense fluorescence and increased amounts were present and graded on the kidney sections of group II rats and with still less involvement in group III animals. In group IV rats the glomerular immune deposits staining for rat IgG increased in fluorescent intensity and amounts but not to the same extent as in group II animals. Mesangial regions of group I and II rat kidneys had just about the same grade involvement for deposition of rat IgM as before at week 8. On the other hand group III rats and IV animals especially, showed very much reduced grades, indicating less IgM depositions in the mesangium. At the end of the experiment kidney sections were also stained for C5b-9. Group II rat kidney sections staining for the membrane attack complex showed faint beaded deposits around the glomerular capillaries. Group III rats had no C5b-9 in their glomeruli, while rats in-group IV had very faint deposits.

Indirect fluorescent antibody test results: It is well established that SPHN, a variant of HN, is initiated and maintained by pathogenic IgG autoantibodies, which are produced following injections of the altered nephritogenic antigen. Progression of the disease consequently is determined by the amount of pathogenic IgG autoantibodies in the circulation. By removing the circulating nephritogenic antigens, a decrease in pathogenic autoantibody response with corresponding improvements in morphological and functional changes are expected.

In this experiment metabolic control rats were not treated and were kept as normal controls. Serum samples analyzed in this group revealed a low level of circulating naturally occurring IgM autoantibodies against the BB regions of the proximal convoluted tubules. In individual rats the level of circulating IgM autoantibodies varied somewhat from one analysis to the next but not to a significant degree.

In group II untreated rats with SPHN the average circulating pathogenic autoantibody response was quite high even at the end of the experiment at 29 weeks. Serum samples analyzed and averaged in groups of 5 rats with low and high G/M ratios did not show downward tendencies in autoantibody mediated immune responses either. At the end of the experiment 80% of the rats still had high autoantibody responses against the target antigen in the kidney. In group III pre- and post-treated SPHN rats, the average calculating pathogenic IgG autoantibodies was low throughout the experiment. By the end of the experiment at 29 weeks, overall it was below 1:10 dilutions in 90% of the rats and in 80% it was 0. In the meantime circulating naturally occurring IgM autoantibodies were elevated and the G/M ratios came to 0 in 80% of the animals. Increased levels of IgM autoantibodies were capable of removing most of the injected chemically altered rKF3 antigen and in this way prevented the development of an elevated pathogenic IgG autoantibody production.

Group IV rats were post-treated with MICs 14 weeks after the induction of the disease. The initial pathogenic IgG autoantibody response during the first 8 weeks or so was high and in many respects similar to responses observed in group II rats. Immediately after the start of treatment it began to decline and by the end of the experiment at 29 weeks the average IgG antibody titer was about 1:10. Eighty percent of the rats had a significantly low IgG autoantibody level and 50% had no circulating IgG autoantibodies. Treatment with MICs at any stage will initiate downregulation of pathogenic autoantibody responses, by removing the altered nephritogenic autoantigen, and result in remission.

Overall progression of autoimmune disease processes in treated and untreated rats: It was observed that pre- and post-treated rats with MICs had by far the least progression and post-treated rats has greatly reduced progression of their diseases, as compared to group II untreated rats' progression. By maintaining a high IgM autoantibody response, the damaging IgG autoantibody production is depressed with consequential slowing down and even termination of the pathogenic autoantibody response. Observing the overall progression of the disease throughout the experiment one can note that group III rats were 16× better off and group N rats 4.5× better off then group II rats. If we observe upward or downward regulation of pathogenic autoantibody responses at a critical point, (8 to 16 week results) measured by G/M ratios in group II and post-treated group N animals before and after treatment then we notice the following. In group II animals there was a 90% upward surge in pathogenic autoantibody response, while in group N rats there was an almost 250% downward response in pathogenic autoantibody production, indicating a surprisingly fast response to treatment. At the end of the experiment at 29 weeks group II rats still manifested a progressive disease (G/M ratio>3), while in both group III and N rats an almost shut down of pathogenic autoantibody response, depicted by very low G/M ratios (0.0636 and 0.105 respectively) were observed.

Table 1 shows kidney sections at 8 and 29 weeks staining by direct fluorescent antibody tests for rat IgG and IgM. Average values are given within the groups. Fluorescent intensity and grade of glomerular lesions of SPHN untreated (group II) and tested (group III and IV) rats are shown. Metabolic controls (group I) are also graded. Each group has 10 rats, except the metabolic controls, having 8 rats.

TABLE 1

| Anti-rat IgG | | | | | | |
|---|---|---|---|---|---|---|
| | | Grade | | | | |
| | | increase due | TBM, | Anti-rat IgM | | |
| Glomerular | Glomerular | to TBM, BB, | BB, BC | Mesangium | Mesangium | Glomerular loop |
| Intensity | Grade | BC staining+ | presence* | Intensity | Grade | Presence |

8 weeks

| | | | | | | | | |
|---|---|---|---|---|---|---|---|---|
| Grp I Metabolic Controls | 0 | 0 | 0 | | 0 | 2.5 | 0.6 | 7/8 |
| Grp II SPHN | 3.8 | 2.8 | 3.2 | (2) | 5 | 3 | 0.7 | 9/10 |
| Grp III SPHN Pre- Post- Tx w/MICs | 0.9 | 0.33 | 0.33 | (10) | 0 | 3.6 | 1 | 7/10 |
| Grp IV SPHN Post- Tx w/MICs | 2.7 | 1.5 | 1.8 | (7) | 3 | 3.2 | 0.7 | 8/10 |

29 weeks

| | | | | | | | | |
|---|---|---|---|---|---|---|---|---|
| Grp I Metabolic Controls | 0 | 0 | 0 | | 0 | 2.5 | 0.7 | 7/8 |
| Grp II SPHN | 3.7 | 3.1 | 3.65 | (1) | 7 | 2.2 | 0.7 | 8/10 |
| Grp III SPHN Pre- Post- Tx w/MICs | 1.6 | 1 | 1 | (7) | 2 | 1.6 | 0.4 | 6/10 |
| Grp IV SPHN Post Tx w/MICs | 3.2 | 1.9 | 2.2 | (3) | 5 | 0.6 | 0.18 | 7/10 |

Abbreviations: BB: brush border, BC: Bowman's capsule, MICs: immune-complex M, SPHN: slowly progressive Heymann nephritis, TBM: tubular basement membrane, Tx: treated, w/: with,
*number of rat kidneys staining one or more of these structures,
+number of rat kidneys below grade 2 glomerular lesions (in brackets)

### Example 2

### Production of a New Model of Slowly Progressive Heymann Nephritis

The invention provides a new model of slowly progressive Heymann nephritis. This novel model of slowly progressive Heymann nephritis (HN) was used to demonstrate the efficacy of the compositions and methods of the invention, as described herein (e.g., see Example 1).

A slowly progressive autoimmune kidney disease was produced in Sprague Dawley rats by subcutaneous injections of a chemically modified kidney antigen (rkF3) incorporated into Alum and Distemper complex vaccine; followed by subcutaneous injections of an aqueous preparation of the same antigen. The kidney disease was induced by the developing pathogenic autoantibodies, following their reaction with the glomerular fixed nephritogenic antigen. Subsequently, immunopathological events lead to chronic progressive immune complex glomerulonephritis and proteinuria.

The slowly developing disease was morphologically and functionally similar to Heymann nephritis. The damage observed in the collected renal samples of experimental animals at 8 weeks and at the end of the experiment by direct fluorescent antibody test, histology and electron microscopy was similar to the typical lesions found in Heymann nephritis rat kidneys but less severe. Animals became proteinuric from 17 weeks onward (instead of the usual 4-8 weeks) and by the end of the experiment at 8 months, 100% of the rats were proteinuric. This new experimental model of autoimmune kidney disease, not complicated by intraperitoneal deposition and retention of Freund's complete adjuvant and renal tubular antigens, allowed us to investigate the pathogenesis of the disease processes from a different aspect and is a better model for the investigation of future treatment options.

Experimental kidney diseases, similar to HN have also been described, such as passive Heymann nephritis and progressive passive Heymann nephritis, see, e.g., Adler (1984) Kidney hit. 26:830-837; Barabas (1974) Br. J. Exp. Pathol. 55:282-290; Barabas (1974) Br. J. Exp. Pathol. 55:47-55; Feenstra (1975) Lab. Invest. 32:235-242. These latter conditions can be produced in susceptible rats by IV injection(s) of heterologous antibodies directed against rat kidney tubular antigens. These latter forms of kidney diseases result from the injected heterologous antibody reacting with glomerular fixed antigens which in turn evoke complement mediated additional injuries in the glomeruli leading to proteinuria. These conditions are not autoimmune diseases and their true relevance in the search for treatment options for naturally occurring autoimmune kidney diseases of man are unclear. However, hastening immune complex disassociation in the glomeruli would be a most desirable achievement, since various events, which are taking place and contribute to glomerular damage in HN and passive HN could be lessened or might even be prevented.

The invention provides a new model of Slowly Progressive Heymann nephritis (SPHN) which closely resembles membranous glomerulonephritis of man in terms of onset and progression. The new approach, for the production of SPHN, was initially investigated in 3 groups of rats at different time intervals and showed reproducibility. In one experiment described herein, this new model of SPHN is compared with control and ITN rats. HN rats became proteinuric at 4 weeks after the induction of the disease while rats in the new experimental model began to be gradually proteinuric from 17 weeks onward. Similarly, pathogenic autoantibody response (as measured by antibody titers in an indirect fluorescent antibody technique) of the new experimental group of rats was greatly reduced during the first 8 weeks of the induction period of the disease.

While HN is an excellent experimental model to study the pathogenesis of an autoimmune kidney disease and morphological and functional changes, which develop, in some situations, it may not be suitable to investigate treatment options because of its rapid and irreversible course. The present invention provides an experimental autoimmune kidney disease model in rats which closely mimics slowly progressive naturally occurring autoimmune diseases of man. The SPHN kidney disease model of the invention can manipulate the immune system in order to slow down or terminate immunopathological events more feasibly than in HN.

Preparation of rat kidney tubular (fraction 3) antigen: Adult normal Sprague Dawley rats were euthanized and immediately bled out, and their blood vessels flushed out with cold physiological saline. Kidneys were collected in a 0.25 mol/L buffered sucrose solution pH 7.4 and homogenized into a relatively fine suspension by a Cyclone virtishear (Virtis). Intracellular components were obtained by subsequent homogenization of the fine renal suspension in a Potter-Elverhjem homogenizer. Rat kidney fraction 3 (rKF3), a mitochondrial rich fraction was obtained by differential centrifugation, as described by Hubscher (1965) Biochem. J. 97:629-642; Pinckard (1966) Clin. Exp. Immunol. 1:33-43; using a Beckman Model J2.21 centrifuge. All procedures were carried out at 4° C.

Preparation of a sonicated ultracentrifuged rKF3 fraction: rkF3 prepared by the technique described above was re-suspended in a 0.25 mol/L buffered sucrose solution and stored at −35° C. till use. The protein concentration of the thawed out rKF3 preparation was determined by a biurette protein estimation, as described by Weichselbaum (1946) Am. J. Clin. Path. Tech. Suppl. 10:40-49. The final protein concentration of the rKF3 preparation was adjusted to 10 mg/ml before being sonicated for 5 minutes at 4° C. using a Branson Sonifier 250 at 60% duty cycle and 8 micro-tip limit. The sonicated preparation was ultracentrifuged at 100,000 G for 1 hour at 4° C. using a Beckman L8-M ultracentrifuge. The supernatant was collected and designated as the u/c rKF3 preparation. Its protein content was adjusted to 4 mg/ml.

Preparation of Azo-u/c rKF3: A method described by Lannigan and Barabas for the preparation of Azo-rKF3 was followed, as described by Lannigan (1969) J. Pathol. 97:537-543. Chemical coupling of the preparation took place in a 0.1 mol/L buffered borax solution at pH 8.2 for 2 hours at 4° C. Diazonium salt was given drop wise to the preparation with continuous stirring and maintenance of pH. The Azo-protein preparation was dialyzed against three changes of PBS pH 7.3 to get rid of uncoupled diazonium salt. The protein content of the preparation was readjusted to 4 mg/mL using polyethylene glycol 8000.

Urinary protein estimation-Twenty-four-hour specimens of urine were collected from individual rats in metabolic cages six times at weekly intervals before the induction of the disease, and then after at weekly intervals throughout the experiment. Urinary protein content was determined by a biurette method, see Weichselbaum (1946) supra, using a Spectronic Genesis 5 Spectrophotometer at 540 nm.

Light Microscopy: Representative samples of kidney specimens were fixed in 10% formol saline and embedded in paraffin and 3 μm thick tissue sections were stained with haematoxylin and eosin, the periodic acid-Schiff reaction and by the methanamine silver stain as described in Barabas and Lannigan (1969) supra.

Electron micoscopy: from representative samples of kidneys 1 mm³ blocks of cortex were fixed and prepared for electro microscopy as in Barabas and Lannigan (1969) supra.

Immunofluorescent Studies:

Direct fluorescent antibody test: Kidney biopsy samples were obtained from each rat, 8 weeks after the induction of the disease and at the end of the experiment at 8 months. Frozen sections were cut at 2-3μ thickness on a Microm HM 500M cryostat and placed into 0.9% saline for 20 minutes before being fixed in Ether:Alcohol (50:50).

Following fixation, sections were washed twice before being stained for rat IgG with suitable dilution of Alexa Fluor® 488-goat anti-rat IgG (H+L) (Molecular Probe) and for rat IgM with suitable dilution of Alexa Fluor® 488 goat anti-rat IgM (μ chain) (Molecular Probe). Alexa Fluor® stained sections were viewed with a Axioscop Zeiss microscope and digital pictures were taken using a digital camera (Diagnostic Instruments inc.) and filed in a Micron computer. Sections obtained from individual rats at the end of the experiment were also stained for C5b-9 with a monoclonal mouse anti-rat C5b-9 IgG antibody and counterstained with suitable dilution of Alexa Fluor® 488 goat anti-mouse IgG (H+L) (Molecular Probe).

Indirect fluorescent antibody test: Blood was collected from individual rats for the estimation of circulating levels of kidney specific autoantibodies. From the three groups of rats blood was obtained for serum samples at 0, 2, 7, 8, 12, 16, 22, 26, 29 and 32 weeks. Sera collected from individual rats were kept at −35° C. until use. Fresh normal rat kidney sections were cut for the study. Dilutions of sera were tested for reactivity against renal tubular cell components for rat IgG and IgM. Titers of sera for reactivity in the IgG and IgM fractions were recorded.

Elution of γ-globulin from diseased rat kidney: Eluted γ-globulin was obtained from homogenized kidneys of classical HN and SPHN diseased rats by an elution procedure using 0.02 mol/L citric acid at pH 3.2, as described, e.g., by Freedman (1960) Arch. Int. Med. 105:224-235; Freedman (1959) Lancet 2:45-46; Lerner (1968) J. Immunol. 100:1277-1287. Following elution for 2 hours, the supernatant containing the γ-globulin was readjusted to pH 7.2 and dialyzed against PBS and then reduced in volume by carborax 8000 to 0.5 cc/2 kidneys. Their protein, contents were determined by the biurette test and their reactivity against structural components of the kidney were observed in an indirect fluorescent antibody test on normal rat kidney sections. Their bioreactivity was tested in normal rats (following removal of one of their kidneys) by IV injection of the eluted γ-globulin. Four days after the injection, animals were sacrificed and their kidney sections were stained for rat IgG and rat IgM. Kidneys removed prior to injection of the eluted γ-globulin were tested similarly.

Grading of rat IgG in the glomeruli of diseased rats: The most abundant component, responsible for the initiation and maintenance of the autoimmune kidney disease was graded by a semi quantitative method as follows:

(1) The intensity of fluorescence was recorded on a 0-4+ scale. The grading of fluorescence was influenced and consequently determined by the amount of fluorescent material (beaded immune-complexes) present in the glomeruli. Fluorescence from 0-4 was observed at a constant microscope setting and differences in fluorescent intensity were recorded.
(2) The amount of fluorescent material (beaded immune-complexes) deposited in the glomerular capillary-loops was also graded from 0-4:

Presence of rat IgG in other than the glomerular capillaries was also observed and recorded in the tubular basement membrane (TBM), tubular cytoplasm, brush borders (BB) of renal tubules and Bowman's capsule.

Presence of rat IgM was also observed and recorded in the mesangium. The beaded mesangial deposits were graded on a 0-4 scale for fluorescent intensity, and also on a 0-4 scale to describe the amount of fluorescent material present in the mesangium (from no deposits in the mesangium to massive depositions of IgM within the mesangial tree). Presence of a minimal amount of IgM in a beaded pattern around the glomerular capillaries, usually with faint fluorescence, was also observed and recorded.

Experimental Design: Individually numbered rats were randomly assigned to the three groups in the experiment.

Metabolic controls: 10 rats were not injected and kept as controls. Animals in this group were bled for sera, their kidneys biopsied and their urine collected and analyzed at the same time intervals as for rats in the test group

Test group I: Slowly Progressive Heymann nephritis (SPHN): 10 rats were subcutaneously injected with u/c Azo-rKF3 in Alum and Distemper complex virus vaccine (Olson et al., 2000). The final volume ratio of Alum to immunogen-Alum mixture was 1:3. The adjuvant antigen mixture was made up as follows:

1 volume of Alum (Imject® Alum by Pierce) was added drop wise to a mixture of 1 volume of Distemper complex virus vaccine (Duramune DA₂P+PV, Fort Dodge, Iowa, USA) and 2 volumes of Azo u/c rKF3 (PBS was added to the antigen to make it up to 2 volumes) and stirred for 30 minutes at RT° prior to injection.

Rats were injected four times with 0.2 mL of antigen adjuvant mixture. On day 0 the mixture contained 160 μg and on days 10, 20, and 35 contained 80 μg antigen. Additional three SC injections of 100 μg of aqueous Azo u/c rKF3 antigen were administered on days 42, 49 and 55. All SC injections were carried out on the dorsal surface between the shoulder blades.

Test Group II: Heymann nephritis (HN) kidney disease: 10 rats were intraperitoneally injected four times with Azo-rKF3 antigen incorporated into FCA. The final volume ratio of FCA to immunogen-FCA mixture was 1:2. The adjuvant antigen mixture was made up as follows: To 2 volumes of FCA (containing 2 mg Mycobacterium Tuberculosis/mL) 1 volume of Azo-rKF3 (24 mg/mL) was added prior to being emulsified, before injection, using an 18 G 2 way needle on syringes. Rats received 0.25 mL of the emulsified preparation containing 2 mg Azo-rKF3 antigen intraperitoneally on days 0, 10, 20 and 35 and on days 42, 49 and 55 a 0.25 mL, aqueous preparation, containing 2 mg Azo-rKF3 was administered subcutaneously between the shoulder blades.

Test Group I and II rats were re-stimulated with 0.2 ml of an aqueous 100 μg Azo u/c rKF3 antigen preparation at 22, 23 and 24 weeks by SC injections.

Results

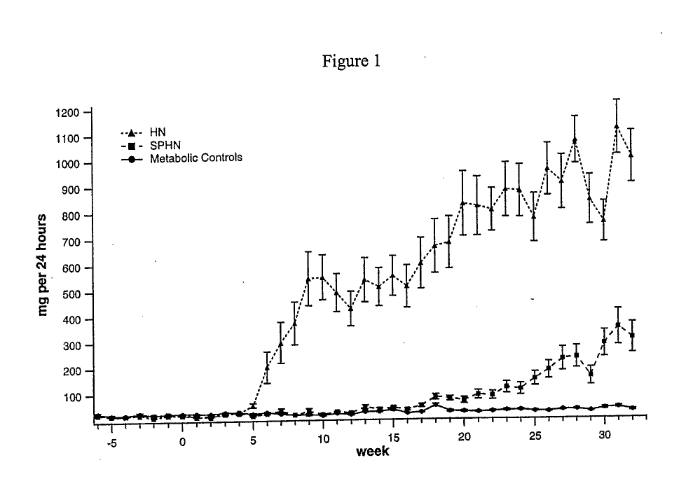

Proteinuria: Throughout the experiment weekly proteinuria estimations were carried out on 24 hour urine samples obtained from individual rats. Proteinuria measurements taken 6 weeks before the start of the experiment from individual rats established a good base line and showed that all the rats in the three groups were aproteinuric. Proteinuria started to develop in test group I SPHN rats 13 weeks after the initiation of the disease and it became slowly progressive from 17 weeks onward. By the end of the experiment at 32 weeks 100% of rats were moderately proteinuric at the same level as FIN rats were approximately 7 weeks after the initiation of their diseases. In test group II HN rats proteinuria started as early as 5 weeks after the first injection of the Azo-rKF3 antigen in FCA and then after it became intense with significantly elevated levels of urinary protein loss. At the end of the experiment 100% of the rats were severely proteinuric. At this stage animals excreted approximately the equivalent of their total serum protein content daily. In addition, in this group most of the rats were thin and fragile looking because of extreme proteinuria associated metabolic dysfunctions. Metabolic controls, kept to see if age related increase in proteinuria during the experiment would occur, showed no significant changes right throughout the 38 week period of testing, as shown in FIG. 1.

Light Microscopy: HxE sections showed a slight increase in cellularity of the glomeruli of test group I and II rats and none in the metabolic controls. Methanamine silver stained kidney sections of proteinuric test group I and II rats revealed thickened glomerular capillaries, prominent mesangial areas and silver positive spikes on the outer surfaces of the thickened glomerular capillaries.

Electron microscopy: Three representative samples of rat kidneys were obtained from each group of rats at the end of the experiment. Group I rats with SPHN showed the characteristic morphological lesions which can be observed in the kidneys of active Heymann nephritis rats. The glomerular basement membranes (GBMs) of the renal glomeruli were irregularly thickened along their entire circumferences due to basement membrane (BM) material growing and encircling partially or completely osmiophilic densities on the epithelial aspect. In relation to the osmiophilic deposits and GBM changes, foot-processes were effaced and the epithelial cell cytoplasm overlaying the lesions manifested osmiophilic areas. Mesangial areas showed electron dense deposits and focally increased mesangial cells. Group II rats with active HN essentially showed similar but more severe changes in the glomerular and related ultrastructure, representing more advanced lesions due to damage caused by increased levels of pathogenic autoantibodies). GBMs were more severely affected by irregularly thickened multilayered and multiprojected BM-like materials encircling and enclosing small to large osmiophilic deposits. Foot-processes in relation to the deposits and GBM changes were effaced and their epithelial cell cytoplasm contained to various degrees osmiophilic stainings. Ultramicrographs of metabolic control kidneys revealed no ultrastructural changes in their GBM and related structures.

Analysis of eluted γ-globulin: Eluted γ-globulin obtained form kidneys of group I and II rats stained the BB regions of the proximal convoluted tubules of normal rat kidney sections in an indirect fluorescent-antibody test. When samples of eluted γ-globulin were injected intravenously into unilaterally nephrectomized rats and biopsied 4 days later, a diffuse fine beaded deposition of rat IgG around the glomerular capillary-loops was observed. Kidney sections obtained from the unilaterally nephrectomized rats prior to injections showed no rat IgG in the glomeruli. Kidney sections obtained from the unilaterally nephrectomized rats prior to injections of eluted γ-globulin were also stained for rat IgM. The same fluorescent pattern of mesangial and fine glomerular capillary-loop staining was observed in the pre- and post-injected kidney samples. Both eluted γ-globulin samples from group I rats with SPHN and group II rats with classical HN gave similar in vivo and in vitro test results.

Direct Fluorescent antibody test results: Table 2, below, shows the direct fluorescent antibody test results on kidney sections obtained from the 3 groups of rats at 8 weeks and at the end of the experiment at 32 weeks.

Metabolic Control Rats: had no IgG localized components in their kidney sections at 8 or 32 weeks. However, all the rat kidney sections had definite depositions of IgM in a beaded pattern in their mesangium focally or diffusely with minimal to moderate involvements and similar fluorescent intensity and grades in all the rats. In addition, a fine beaded staining of the glomerular capillary-loops was also noted, indicating deposition of rat IgM at these sites.

Test group I SPHN rats: showed IgG deposits in the glomeruli, brush border associated regions and in the TBM. The most definite presence of rat IgG was observed in the glomerular capillary blood vessels in a beaded pattern. From sparse small beaded deposits to large confluent multilayered beaded deposits were observed at these sites, graded and recorded at 8 and 32 weeks. In addition BB region of an occasional proximal convoluted tubule stained also but with a fainter fluorescence and more so at 8 weeks then at the end of the experiment. TBM stained with a beaded pattern of patchy distribution. This pattern of fluorescence was observed during the early period at 8 weeks and less frequently at 32 weeks. IgM was present in the mesangium on every rat kidney section with a beaded pattern at 8 and 32 weeks and its presence and distribution did not seem to be effected by the disease state. It was also observed at 8 and 32 weeks around the glomerular capillary blood vessels with a beaded rather faint fluorescence on most rat kidney sections.

Test group II HN rats: Rat IgG was observed in the glomerular capillary blood vessels with heavy deposits in a granular often multilayered pattern early on at 8 weeks and much more pronounced with confluent large deposits at the end of the experiment. Tubular BB regions stained with diffuse intense staining at 8 weeks and at the end of the experiment tubular BB staining was more intense and diffuse showing increased damage overtime. Tubular basement membranes stained with a beaded pattern for rat IgG quite wide spread on kidney sections at 8 weeks and at the end of the experiment stained similarly. Bowman capsules stained for IgG in parts with beaded deposits on a few rat kidney sections at the end of the experiment only. Rat IgM was found in the mesangium and in the glomerular capillary-loops as described for metabolic control and SPHN rats. In addition 5 rats at 8 weeks showed minimal but definite staining of BB regions for IgM. At the end of the experiment 7 rats showed diffuse cytoplasmic staining of the renal proximal tubules for IgM. One rat showed diffuse staining of the glomerular capillaries in a beaded pattern for IgM.

All the rat kidney sections were stained by the sandwich technique for the membrane attack complex C5b-9. The glomerular capillary-loops of group I and II rats' kidney sections stained strongly with a diffuse beaded pattern for C5b-9 at the end of the experiment. Kidney sections from metabolic rats did not stain.

Indirect Fluorescent Antibody Test Results

Metabolic control: Sera of normal rats analyzed by an indirect fluorescent antibody test before and right though the experiment, revealed a low level of circulating naturally occurring IgM autoantibodies which were directed against the BB regions of the proximal convoluted tubules of normal rat kidney sections. The pattern of immunofluorescence at the periluminal region of the proximal convoluted tubules was composed of an intricate fine linear staining. Most often the typical tubular staining for rat IgM was not diffuse but randomly distributed on the kidney sections involving one or more tubules at any one location. In individual rats, the level of the circulating IgM autoantibody varied somewhat from one analysis to the next but not to a significant degree. Sera samples did not have antibody activities against normal rat kidney section components by rat IgG antibodies.

Test group I SPHN: A moderate IgG antibody response to renal tubular epithelial cell components was present within 2 weeks; and a very much-increased response was recorded by 7 weeks that continued into the 8^{th} and 12^{th} weeks. From 16 weeks onward there was a gradual decline in autoantibody response, which was boosted by the three times injected aqueous Azo u/c rKF3 antigen from 22 weeks. Throughout the study, tubular fluorescence by the indirect fluorescent antibody tests was diffuse involving practically all the tubules by staining the BB related regions with a typical wide staining pattern. IgM antibody response to tubular BB related areas have increased on average four times above normal physiological range right from the beginning to the end of the experiments. Following Azo u/c rKF3 antigen injections from 22 weeks, there was an increase in IgM autoantibody response also.

Test group II HN rats: Anti-tubular BB IgG antibody response in this group of rats was swift and by two weeks after the induction of the disease the average antibody titer was over 1000. By 7 weeks the antibody titer was at its highest, being just over 30,000 and then after at 8, 12 and 16 weeks it was still high but with declining values reaching relatively low but still significant levels at 32 weeks (FIG. 14). In this group every rat had high pathogenic autoantibody response. Just as in test group I rats, typical BB staining pattern was observed by the indirect fluorescent antibody tests. Re-stimulation of these rats at 22 weeks with aqueous Azo-rKF3 antigen did not increase antibody response. IgM antibody response to tubular BB related areas on the average was about 100× above normal physiological values by week 7, and then after remained still very high on weeks 8 and 12 and somewhat in a descending order than after. At the end of the experiment IgM anti-tubular antibody response on the average was still 10× above normal physiological values. The immunofluorescence staining of renal proximal tubules for IgM was exaggerated and showed both diffuse granular cytoplasmic and rough multilinear staining patterns.

Discussion: The invention provides methods for making and using a novel autoimmune kidney disease, morphologically and functionally similar to HN, produced in Sprague Dawley rats by a novel technique that is a minimally invasive procedure. In the experiments described herein animals received SC injections of a low dose of chemically modified renal tubular antigen incorporated into Alum and Distemper complex vaccine, followed by SC injections of the same antigen in an aqueous medium.

The developing disease was slowly progressive. Minimal proteinuria started at 13 weeks and frank proteinuria began from 17 weeks onward, and at the end of the experiment at eight months 100% of rats were proteinuric. Early kidney biopsy samples obtained at 8 weeks for direct fluorescent antibody studies showed immune deposits staining for rat IgG around the glomerular capillaries in a beaded pattern.

At the end of the experiment, kidney tissue samples obtained for fluorescent antibody tests, histology and electron microscopy showed the typical changes of HN. By direct fluorescent antibody tests diffuse beaded depositions of immune complexes were observed around the glomerular capillaries staining for rat IgG and C5b-9; and BB regions of the proximal convoluted tubules and TBMs also stained as shown in Table 2. Histology revealed, on silver methanamine stained kidney sections of proteinuric rats, thickened glomerular capillaries with silver positive spikes around their outer circumferences and prominent mesangium.

Electron microscopy showed large osmiophilic deposits embedded in the irregularly thickened outer surface of the GBM. In addition, effacement of the foot-processes in relation to the deposits was also observed.

By an indirect fluorescent antibody test we investigated the presence of circulating pathogenic and non-pathogenic autoantibodies. It was shown that at the beginning of the experiment, especially as the injection of the alum incorporated antigen continued, that the level of circulating pathogenic autoantibodies were high and as the experiment progressed its level dropped. At the end of the experiment a low level of circulating autoantibody directed against tubular BB related antigens was still detectable. These autoantibodies (which were produced as a result of “unusual presentation” of self-like antigens) were responsible for the initial phase events. Accordingly the developing autoantibodies reacted with the glomerular-bound nephritogenic antigens and formed immune complexes. If no additional chemically altered antigen would have been made available as the experiment progressed, then it would be reasonable to assume that no more circulating pathogenic autoantibodies could have formed and equally no more antigen would have been made available for the ever expanding and growing immune complexes (made up of nephritogenic antigen, autoantibody and complement components) on the epithelial aspect of the GBM.

Table 2 shows kidney sections of individual rats stained in the 3 experimental groups by the direct immunofluorescence technique for rat IgG and IgM. Average fluorescence intensity and average grades within individual groups of rats as well as presence or absence of kidney tissue localized components at 8 and 32 weeks are shown.

TABLE 2

| Anti-rat IgG | | | | Anti-rat IgM | | |
|---|---|---|---|---|---|---|
| Glomerular | Glomerular | BB | TBM | Mesangium | Mesangium | Glomerular |
| Intensity | Grade | presence | presence | Intensity | Grade | Loop Presence |

8 weeks

| | | | | | | | |
|---|---|---|---|---|---|---|---|
| Metabolic Control | negative | negative | negative | negative | 2+ | 0.6 | 9/10 |
| HN | 4+ | 3.9 | 10/10 | 10/10 | 2.5+ | 0.9 | 8/10 |
| SPHN | 3.2+ | 2 | 6/11 | 6/11 | 2.8+ | 0.9 | 10/11 |

32 weeks

| | | | | | | | |
|---|---|---|---|---|---|---|---|
| Metabolic Control | negative | negative | negative | negative | 3.2+ | 0.7 | 8/10 |
| HN | 4+ | 4 | 10/10 | 8/10 | 3.2+ | 1 | 8/10 |
| SPHN | 4+ | 3 | 3/11 | 3/11 | 3+ | 0.7 | 5/11 |

BB: Brush Border, TBM: Tubular basement membrane, HN: Heymann nephritis, SPHN: Slowly Progressive Heymann nephritis

### Example 3

### Production of Heymann Nephritis by a Chemically Modified Renal Antigen

This example describes the production of Heymann nephritis (HN) by a chemically modified renal antigen and demonstrates that a chemically modified nephritogenic antigen in an aqueous media, without the use of any adjuvants, is capable of initiating a pathogenic autoimmune response in a susceptible strain of rats.

An autoimmune kidney disease, morphologically and functionally similar to Heymann nephritis (HN), was induced in mature male Sprague Dawley rats by repeated weekly IP injections of a chemically modified Azo ultracentrifuged (u/c) rKF3 antigen in an aqueous media, see Example 2, above.

The experiment was terminated 15 weeks after the first injection of the chemically altered antigen. Serum samples collected and analyzed by an indirect fluorescent antibody test on normal rat kidney sections during the course of the experiment showed a gradual rise in the circulating pathogenic autoantibodies which were directed against the proximal tubular brush border regions. Proteinuria was present and significantly increased in the urine of a few rats. The developing immune-complex glomerulonephritis revealed the typical HN kidney disease lesions in 70% of the rats by histological, direct fluorescent antibody and electron microscopical examinations.

Control rats injected similarly with the same chemically unmodified antigen did not develop the characteristic morphological and functional changes.

These data describe for the first time that the autoimmune kidney disease designated as active HN can be produced by the administration of a chemically altered renal antigen in an aqueous solution and not by the usual presentation of the nephritogenic renal antigen in an adjuvant.

Animals: Adult over one year old male Sprague Dawley rats were used in the experiment. The individually numbered and randomly assigned rats to the control and test groups were obtained from a local breeding colony. All the invasive experimental procedures were carried out on Isoflurane anaesthetized rats. At the end of the experiment animals were euthanized by IP injections of Euthanyl (MTC pharmaceuticals) (180 mg/kg body weight).

Experimental Design

Control rats: 15 rats were used in this group. These animals were injected intraperitoneally with 100 μg of an unmodified sonicated ultracentrifuged rKF3 preparations in 0.2 ml PBS pH 7.3 at weekly intervals.

Test rats: 8 rats were injected intraperitoneally with 100 μg Azo-sonicated ultracentrifuged rKF3 preparation in 0.2 ml PBS pH 7.3 at weekly intervals.

Blood samples were collected for sera from each animals before the start of the experiment and at 2, 4, 6, 12 and 15 weeks.

Kidney biopsy samples were obtained from each rat for analysis by direct fluorescent antibody tests before the experiment started and from a few rats two weeks into the investigation and from all the rats at the end of the experiment. At the end of the investigation indirect fluorescent antibody test was carried out on each serum sample collected throughout the experiment. In addition each rat kidney sample was also examined by histological techniques of specifically stained tissue sections. By electron microscopy all the test group kidneys were examined but only a few kidney specimens in the control group. The experiment was terminated at 15 weeks.

Urinary protein estimation: Before the start of the experiment 24 hours specimens of urine were collected from individuals rats three times at weekly intervals in metabolic cages and then after twice during the experiment. Urinary protein estimation was carried out on 0.5 ml urine specimens by a biurette method using a spectronic Genesis 5 Spectrophotometer at 540 nm. Daily proteinuria was calculated and expressed as mg/day protein loss per 100 gm body weight.

Preparation of rat kidney tubular fraction 3 (rKF3) antigen: Kidneys were obtained from euthanized adult Sprague Dawley rats following exsanguinations and washing out their blood vessels with 4° C. PBS pH 7.2. The kidneys were collected in a 4° C. 0.25 mol/L buffered sucrose solution pH 7.4 and washed several times in the buffer to get rid of blood components. Kidney samples were homogenized into a fine suspension by a Cyclone Virtishear (Virtis) and the intracytoplasmic components were released into the sucrose buffer solution using a Potter-Elverjhem Teflon homogenizer. Rat kidney fraction 3, designated as rKF3 was obtained by differential ultracentrifugation [17] using a Beckman Model L-2 ultracentrifuge. All procedures were undertaken at 4° C. The protein concentration of the rKF3 preparation was determined by the biurette technique [16] and adjusted to 30 mg/ml before storing it at −35° C.

Preparation of sonicated u/c rKF3: A 10 mg/ml rKF3 preparation in 0.25 mol/L buffered sucrose solution pH 7.2 was sonicated for 5 minutes at 4° C. using a Branson sonifier 250 at 60% duty cycle at 9 micro-tip limit. The sonicated preparation was ultracentrifuge at 100,000 G for 1 hour at 4° C. using a Beckman L8-M ultracentrifuge. The resulting supernatant was designated as the u/c rKF3 preparation and its protein content was adjusted to 4 mg/ml before storing it at −35° C. till further use.

Preparation of Azo sonicated u/c rKF3: A method described by Lannigan, et al. for the preparation of Azo-rKF3 was employed (Lannigan, et al., Some experimental models of the nephritic syndrome. In: Bajusz E., Jasmin G, eds. Meth Achievement Experimental Pathology. New York: Karger, Basel, 1969). The chemical coupling of the sonicated u/c rKF3 preparation took place in a 0.1 mol/l buffered borax solution at pH 8.2 for 2 hours at 4° C. At continuous stirring diazonium salt was given to the preparation dropwise while pH was maintained at 8.2. The developing yellow Azo-protein preparation was dialyzed against several changes of PBS pH 7.2 to eliminate uncoupled diazonium salt. The protein content of the Azo-protein compound was readjusted to 4 mg/ml using polyethylene glycol 8000.

Histology: Cortical portions of kidney samples were fixed in 10% neutral buffered formation and paraffin embedded sections were cut and stained with haematoxylin and eosin (H&E), periodic acid Schiff (PAS) and periodic acid Schiff methenamine (PASM) stains. The sections were examined by a Zeiss Axioscope Microscope.

Electron microscopy: Representative samples of kidneys 1 mm3 blocks of cortex were fixed in 2.5% cacodylate buffered glutaraldehyde for 2 hours, post-fixed in Caulfield's osmium tetroxide solution and embedded in Epon. Thin sections, containing glomeruli, were stained with uranyl acetate and lead citrate. Ultrathin sections were examined using a Hitachi H600 electron microscope.

Immunofluorescent studies: Frozen sections of cortical renal tissue samples were cut at 2-3μ thickness with a micron HM 500M cryostat and placed in a coplin staining jar with 0.9% saline for 10 minutes before being fixed in Ether:Alcohol (50:50) for 2 minutes and then washed again.

Direct fluorescent antibody test: Ether: Alcohol fixed sections were incubated in a wet box for 30 minutes with suitable dilutions of Alexa Fluor® 488-anti-rat IgG (H+L) and Alexa Fluor® 488 goat anti-rat IgM (pt chain) (Molecular Probe). Following incubation with the labeled antibodies, sections were washed in two changes of saline prior to mounting with glycerol/PBS (50:50).

Sandwich Technique: Sections obtained from individual rats at the end of the experiment were also stained for C5b-9 with monoclonal mouse anti-rat C5b-9 IgG antibody and counter stained with a highly cross-absorbed Alexa Fluor® 488 goat anti-mouse IgG (H+L) (Molecular Probe) at 4000-dilution. Similarly rat kidney sections were also stained for rat C-3 with a rabbit anti-rat C-3 IgG antibody and counter stained with Alexa Fluor® 488 goat anti-rabbit IgG (H+L) (Molecular Probe).

Indirect fluorescent antibody test: Dilutions of serum samples from individual rats obtained before, during and at the end of the experiment were tested for reactivity against renal tubular components on normal rat kidney sections in the rat IgG and rat IgM fractions. After incubating with dilutions of sera, appropriate sets of sections were stained with Alexa Fluor®488 goat anti rat IgG (H+L) and Alexa Fluor® 488 goat anti-rat IgM (μ chain) (Molecular Probe). Appropriate controls were included in the fluorescent antibody tests.

Elution of γ-globulin from diseased rat kidneys: Eluted γ-globulin was obtained from suitably prepared glomerular preparations by an acid elution technique using 0.02 mol/L citric acid at pH 3.2, see, e.g., Freedman (1959) Lancet 2:45-6; Freedman (1960) Arch. Int. Med. 105:224-235. The elution process took 2 hours. After centrifugation, the supernatant containing the eluted γ-globulin was readjusted to pH 7.2 and dialyzed against three changes of PBS and then reduced in volume by Carbovax 8000 to 0.5 ml/2 kidneys. The protein content of the concentrated samples were determined by the biurette test (see, e.g., Weichelbaum (1946) Am. J. Clin. Path. Tech. Suppl. 10:40-49) and their reactivity against normal rat kidney components were observed in an indirect fluorescent antibody test on normal rat kidney sections. The bioreactivity of the eluted γ-globulin was tested following its IV injection into a unilaterally nephrectomized Sprague Dawley rat. Four days after the injection the rat was euthanized and its kidney sections stained for rat IgG and rat IgM. Kidney sections prior to injection of the eluted γ-globulin were tested similarly.

Grading of glomerular localized autologous components: The most abundant glomerular localized component, responsible for the development of the disease, was rat IgG The intensity of the fluorescence was determined by the amount of fluorescent material (the beaded glomerular immune-complexes) and it was graded on a 0-4+ scale by a semi quantitative method at a constant microscope setting. The amount of fluorescent material in the glomeruli was also graded on a 0-4+ scale (see Example 2). Grade 0 lesion had no glomerular deposits, while grade 4+ lesion had diffuse large often multilayered beaded deposits around the glomerular capillaries. In between grades were determined according to set values.

Presence of rat IgG was also noted and recorded in the tubular basement membrane (TBM), tubular cytoplasm, brush border (BB) and Bowman's capsule. Presence of rat IgM was observed and recorded in the mesangium. The fluorescent intensity and the amount of fluorescent material in the mesangium was graded on a 0-4+ scale. A minimal amount of IgM with a faint beaded pattern was also present in the glomeruli of the pre- and post injected animals' kidney samples and recorded.

Results

Proteinuria: Three weekly proteinuria results obtained from individual rats prior to experiment revealed low levels of normal proteinuria values in both groups of rats (12 mg/day/100 gr body weight). Two rats in the test group became highly proteinuric with 140 and 290 mg/day/100 gr body weight respectively and non in the control group by the end of the experiment.

Light Microscopy: Test group rats' kidney sections showed slight increase in cellularity on H&E sections. PAS stained kidney sections revealed in both test and control animals pathological sclerosing glomerular lesions characteristically found in older rats. Methanamine silver stained kidney sections of the two proteinuric test group rat's showed prominent mesangial areas and thickened glomerular capillaries with multilayered silver-positive spikes on their outer circumferences. Four non-proteinuric test group rats showed similar but milder involvements of the glomeruli with occasional silver positive spikes on their outer walls. Control rats did not have the typical lesion.

Electron microscopy: Severe ultrastructural changes, typically observed in active HN rat kidneys, were observed in the glomeruli of the two proteinuric rats. The massively and irregularly thickened glomerular basement membrane (GBM) towards the epithelial aspect of the glomeruli partially or completely surrounded small to large osmiophilic deposits. In relation to the GBM changes and foot-process fusions the epithelial cell cytoplasm manifested osmiophilic areas with the same degree of intensity as the deposits themselves. An additional 4 test rat kidneys manifested a milder form of active HN lesions. In these rats a patchy irregularly thickened GBM with smallish osmiophilic deposits were observed. Epithelial cell foot-processes were only fused in relation to the GBM found deposits and were preserved in many areas where deposits were not present. Two rats in the test group had no HN kidney lesions and none of the rats in the control group.

Direct Fluorescent antibody test results: Table 3 shows the presence or absence of renal tissue localized rat IgG and IgM antibodies at 0 and 15 weeks. Control rats at 0 and 15 weeks showed no rat IgG in the glomeruli or in related structures. On the other hand mesangial beaded depositions of rat IgM, most often with intense fluorescence, was observed on the kidney sections of all the rats. A faint beaded deposition of rat IgM, was also observed around the glomerular capillaries. C5b-9 was present in the mesangium minimally, in the end of the experiment kidney samples, with a faint beaded immunofluorescence pattern but not C-3.

Test group rats at 0 week showed the same fluorescent antibody test results as the controls. Five out of eight rats biopsied for cortical kidney samples, three weeks after the first injection of the modified renal antigen, showed in one kidney biopsy sample a fine diffuse beaded deposition of rat IgG around the glomerular capillaries and the same sample showed staining of the brush border (BB) regions of renal tubules at places. The other three kidney biopsy samples showed barely detectable fine beaded staining of the glomerular capillary-loops for rat IgG and one sample was negative.

At the end of the experiment six out of the eight rats developed immune complex glomerulonephritis characterized in five rats by a heavy beaded deposition of rat IgG around the glomerular capillary-loops and in one rat by a similar but lighter deposition. Characteristic localization of rat IgG observed in classical HN, in the BB region and in the TBM with a beaded pattern was also observed in the kidneys of three rats and occasional segments of the Bowman's capsule were minimally stained with a beaded pattern in 2 rats' kidneys. Kidney sections were also stained, for C5b-9 and C-3 at the end of the experiment. Three rats' kidney sections, stained, with various degrees of intensity the glomerular capillaries for C5b-9 with a beaded pattern and all the other rat kidney sections showed minimal beaded mesangial stainings. Four rat kidney sections stained with a fine diffuse beaded pattern for C-3. Rat IgM was present with increased amounts in the mesangium with a beaded pattern at the end of the experiment and glomerular capillaries stained with a faint fine diffuse beaded pattern of fluorescence.

Indirect Fluorescent antibody test results: Control and test rats' sera, before the start of the experiment showed a low level of circulating naturally occurring IgM autoantibody, directed against the BB region of the proximal convoluted tubules with a fine linear pattern of fluorescence, on normal rat kidney sections. No circulating rat IgG antibody activity was observed against normal rat kidney tissue components. During the course of the experiment serum samples were obtained from individual rats six times.

Control rats injected with the native nephritogenic antigen did not develop pathogenic IgG autoantibodies, but produced a slightly elevated level of IgM autoantibodies against renal tubular antigens. Six out of the eight rats, injected with the chemically modified nephritogenic antigen developed increasing levels of pathogenic IgG autoantibodies as the disease progressed against the BB associated regions of the renal proximal convoluted tubules. Non-pathogenic IgM autoantibody level also increased much more than in the control rats, signifying increased stimulation of the IgM producing cell lines.

Analysis of the eluted γ-globulin: Eluted γ-globulin was obtained from the test group of rats only. It was tested in an indirect fluorescent antibody test at a protein concentration of 5 mg/ml. It reacted with the BB regions of the proximal convoluted tubules up to 1:40 dilutions. When a 0.5 ml sample of the eluted γ-globulin was injected intravenously into a unilaterally nephrectomized rat and biopsied four days later, a diffuse fine beaded deposition of rat IgG was observed around the glomerular capillaries in a direct fluorescent antibody test. Pre-injection kidney sections did not stain for rat IgG but stained for rat IgM as already described for the controls.

Discussion: The methods of the invention produced HN in a group of rats by repeated injections of a chemically modified nephritogenic antigen in an aqueous solution. The developing disease, was characterized by immune-complex depositions in the glomeruli and proteinuria in the most severely effected rats. This autoimmune disease was initiated and maintained by pathogenic autoantibodies. Those control rats, which were injected with the same, but chemically unaltered antigen did not develop the autoimmune kidney disease.

These experiments demonstrate that a self-antigen has to be sufficiently altered before it is recognized as foreign by appropriate immune cells prior to a pathogenic immune response to occur. This point is well illustrated in patients treated with certain drugs and subsequently develop lupus-like syndromes, see, e.g., Jiang (1994) Science 266:810-813; Rich (1996) Postgrad. Med. 100:299-298; Totoritis (1985) Postgrad. Med. 78:149-161; Yung (1995) Lab Invest. 73:746-759. When the medication is withdrawn, the lupus like symptoms disappear. In a susceptible patient, the drug-induced lupus must have started by the active principle or by a break down product of the drug being able to alter sufficiently appropriate intracytoplasmic antigen/s and start up the immunopathological events. In certain individuals sunshine or extreme cold can also produce lupus-like syndromes and once again by non-exposure to these hazards, the symptoms disappear.

These experiments also demonstrate that animals repeatedly injected with the modified renal antigen produced elevated levels of both pathogenic and non-pathogenic autoantibodies during the experiment; while those rats which were injected with the native renal antigen produced slightly increased levels of naturally occurring non-pathogenic IgM autoantibodies only.

The present experiments, and the methods and compositions of the invention, clearly demonstrate that autoimmunity can serve a beneficial purpose by assisting in the efficient removal of released intracytoplasmic components by IgM autoantibodies. These experiments also demonstrate that inappropriate presentation of a sufficiently altered self-antigen can under certain circumstances initiate a harmful pathogenic autoantibody response and cause disease.

Table 3 shows kidney sections stained by the direct immunofluorescence technique showing the presence/absence of tissue localized rat IgG and rat IgM on 0 and 15 weeks.

TABLE 3

| Anti-rat IgG | | | | Anti-rat IgM | | |
|---|---|---|---|---|---|---|
| F1* | Grade of | | | F1* intensity of | Grade of | |
| intensity of | glomerular | Presence | Presence | mesangial | mesangial | Presence in |
| deposits | lesion | at BB | at TBM | deposits | deposits | glomeruli |

0 week

| | | | | | | | |
|---|---|---|---|---|---|---|---|
| Controls | −ve | −ve | −ve | −ve | 3+ | 0.7 | Faint diffuse beaded |
| Tests | −ve | −ve | −ve | −ve | 3+ | 0.6 | Faint diffuse beaded |

15 weeks

| Controls | −ve | | −ve | | −ve | −ve | 3.5+ | 1 | Faint diffuse beaded |
|---|---|---|---|---|---|---|---|---|---|
| Tests | 5/8 | 4+ | 5/8 | 3 | 3/8 | 3/8 | 3.5+ | 2 | Faint diffuse beaded |
| | 1/8 | 2+ | 1/8 | 1.5 | | | | | |
| | 2/8 | −ve | 2/8 | −ve | | | | | |

BB = brush border, F1* = fluorescence; TBM = tubular basement membrane
15 rats were included in the controls and 8 rats in the tests.

### Example 4

### Downregulation of Pathogenic Autoantibody Responses in Slowly Progressive Heymann Nephritis Rats Repeatedly Stimulated with a Nephritogenic Antigen

These experiments demonstrate the efficacy of the methods and compositions of the invention, In particular, they demonstrate downregulation of pathogenic autoantibody responses in slowly progressive Heymann Nephritis rats repeatedly stimulated with a nephritogenic antigen.

An autoimmune kidney disease called Slowly Progressive Heymann Nephritis (SPHN) (see Example 2, above) was induced in 3 groups of rats by repeated SC injections of a small dose of an azo ultracentrifuged (u/c) rat kidney fraction3 (rKF3) preparation incorporated into Alum and Distemper complex virus vaccine. The developing kidney disease was characterized by immune-complex glomerulonephritis (ICGN) and slowly progressive proteinuria. It was initiated and maintained by the developing pathogenic autoantibodies, which were directed against the nephritogenic antigen residing in the glomeruli and bush-border (BB) regions of the proximal convoluted tubules of rat kidneys. To make the disease more progressive we re-injected all the SPHN rats three times with the aqueous preparation of azo u/c rKF3 24 weeks after the initiation and 14 weeks following the last injection of the nephritogenic antigen. Group II rats received the disease producing injections and no other treatment, group III animals in addition were pre- and post-treated with immune complexes (ICs) made up of rKF3 antigen and rat anti-rKF3 IgM antibody in antigen excess, designated as “MICs”; and group IV rats were injected with MICs from 7 weeks after the induction of the disease.

The level of circulating specific IgM autoantibody in those animals which were injected with MICs became elevated and the pathogenic IgG autoantibody response reduced. This antigen specific down regulatory effect, in the MICs treated animals, was still maintained by elevated levels of circulating IgM autoantibodies following re-stimulation with the aqueous azo u/c rKF3 antigen. At the end of the experiment 60% of the MICs treated group III and N rats had no pathogenic IgG autoantibodies in their circulation, while all the untreated group II rats had them. The level of circulating pathogenic IgG autoantibody in the untreated SPHN rats was high throughout the experiment and became somewhat more elevated in most of the rats following restimulation with the nephritogenic antigen. In these animals disease progression was also more evident.

These experiments demonstrate that a pathogenic IgG autoantibody induced experimental autoimmune kidney disease process can be down-regulated both early on and even during the chronic progressive phase of the disease by an antigen-specific treatment protocol, using tailor made MICs (exemplary compositions of the invention). It seems the developing IgM autoantibodies, by removing or blocking nephritogenic antigens can prevent stimulation of the IgG autoantibody producing cell lines to make more disease causing pathogenic autoantibodies.

Animals: Randomly assigned and numbered two month old male Sprague Dawley rats, obtained from the local breeding colonies were used in the experiment. All the invasive procedures were carried out on Isoflurane anaesthetized rats and at the end of the experiment at 32 weeks rats were euthanized by IP injections of Euthanyl (MTC pharmaceuticals) (180 mg/kg body weight).

Experimental Design

Group I. Metabolic controls: 10 rats were not injected or treated but proteinuria studies, blood collections for sera and kidney samples were obtained to study changes.

Group II. Slowly progressive Heymann nephritis (SPHN): 10 rats received repeated SC injections of 0.2 ml azo-u/c rKF3 antigen incorporated into Alum and Distemper virus vaccine by a method previously described, see above examples. On day 0, 160 μg antigen containing mixture on 10, 20, and 35, 80 μg and on days 42, 49 and 55, 100 μg aqueous azo u/c rKF3 antigen.

Group III Pre- and Post-treated rats with SPHN: 10 rats were pre-treated with MICs as follows. On days −22, −18, −14, −8 and −3, prior to the induction of the disease by the protocol described for group II rats, animals received IP injections of MICs containing 60 μg rKF3 and 150 μg rarKF3 IgM in 0.2 ml PBS and then after weekly.

Group IV Post-treated rats with SPHN: SPHN was induced in 10 rats by the same protocol as described for group II rats. Seven weeks after the induction of the disease rats were treated by weekly IP injections of MICs containing 60 μg rKF3 and 150 μg rarKF3 IgM in 0.2 ml PBS.

Rats in groups II, III, and IV were re-stimulated on week 22 three times at 5 day intervals with 100 μg aqueous azo u/c sonicated rKF antigen.

Preparation of azo u/c sonicated rKF3 antigen: Homogenized normal rat kidneys in 0.2M sucrose pH 7.4 were used to prepare rKF3 by differential centrifugation. rKF3 preparation was sonicated and ultracentrifuged at 100,000 G for 1 hour to obtain the u/c sonicated supernatant preparation [B+C+D+C]. It was chemically modified to obtain azo u/c sonicated rKF3 preparation using diazuium salt in a 0.1 mol/L buffered borax solution at pH 8.4. The protein content of the azo-protein conjugate was adjusted to 4 mg/ml.

Preparation of rat anti-rKF3 IgM: A low level of circulating naturally occurring IgM autoantibody can be stimulated to obtain a higher IgM autoantibody response against the BB-regions of renal proximal tubules. We injected adult Wistar rats weekly by IP administration of 100 μg rKF3 antigen in PBS for 4 weeks. Four days after the last injection of the antigen individual rats were bled for sera and tested for antibody activity by the indirect fluorescent antibody test on normal rat kidney sections for rat IgG and rat IgM antibody activity against the BB associated antigen. Sera with high IgM antibody tire (1:70-1:180) were pooled, aliquotted and stored at −35° C. until use. Additional rarKF3 IgM antibody was obtained as needed following restimulation of the same rats.

Preparation of rKF3×rarKF3 IgM immune-complexes designated as MICs: MICs for 10 rats were prepared fresh each time as follows: To 600 μg rKF3, 1500 μg rarKF3 IgM (2000 μg IgM/ml of serum) with 1:120 antibody activity to BB antigens was given and made up to 2 ml with PBS. The mixture, at slight antigen excess, was incubated and rotated at RT° for 30 minutes prior to 0.2 ml IP injections of test rats at the appropriate times.

Urinary protein estimation: Twenty-four hours of urine samples were collected from individual rats in metabolic cages. Eight weekly urine samples were obtained and analyzed for baseline values before the start of the investigation then after weekly samples were collected and analyzed to observe differences due to treatment and no treatment. Urinary protein values were determined on 0.5 ml samples of urine by a biurette method using a Spectronic Genesis 5 Spectrophotometer at 540 nm (see above).

Histology, electron microscopy and direct fluorescent antibody test on renal cortical samples: 10% neutral-buffered formalin fixed renal cortical samples were embedded in paraffin and 3 μm thick sections were cut and stained with hematoxylin and eosin and methenamine silver stain [B+L]. Electron microscopical examination of suitably fixed and stained ultra thin sections of renal cortical specimens were examined with a Hitachi H600 electron microscope as described earlier (B+C+B+L). Three μm thick fresh kidney cortical specimens, from individual rats, suitably processed were stained for the presence of rat IgG and rat IgM with appropriate dilutions of Alexa Fluor® 488 labeled goat anti-rat IgG (H+L) and goat anti-rat IgM (u chain) (Molecular Probe) at 8 weeks and the end of the experiment. Kidney sections were also stained for C5b-9 with a monoclonal mouse anti-rat C5b-9 IgG antibody and counter stained with a suitable dilution of Alexa Fluor® 488 highly absorbed goat anti-mouse IgG [H+L] (Molecular Probe) at the end of the experiment only (B+L+B+L). See Table 4.

Indirect fluorescent antibody test and grading of the glomerular lesions resulting from the deposition of rat IgG in the glomerulus: Rat IgG and IgM antibody titers of serum samples of individual rats directed against normal rat kidney tubular components were determined and expressed as reciprocals of the last dilutions of sera giving positive results. The intensity of fluorescence and the amount of fluorescent material in the glomerular localized immune complexes was graded on a 0-4+ scale as previously described (B+C+B+L). The presence of rat IgG in the tubular basement membrane, brush-border region of the proximal tubules and Bowman's capsules were also recorded and similarly rat IgM found in the mesangium and glomerular capillaries was recorded and graded.

Progression of the disease: The disease progression was determined by calculating and plotting G/M ratios. G/M ratio is a number procured by dividing the reciprocal number of the highest IgG autoantibody titer with the reciprocal number of the highest IgM autoantibody titer which were obtained in the indirect fluorescent antibody test. G/M ratios of the disease producing IgG autoantibody negative rats is 0. G/M ratios were determined in individual rats' sera collected at 2, 7, 8, 12, 16, 22, 26, 29 and 32 weeks. Average G/M ratios within groups of rats were determined and also the same ratios in 5 rats with the lowest and in 5 rats with the highest values were calculated and plotted.

Proteinuria: Eight weekly collections of urine samples were analyzed from individual rats for proteinuria to establish representative base line values prior to the induction of the kidney disease. Then after metabolic control rats provided the continuous base-line proteinuria values during the experiment. Towards the end of the experiment, the average proteinuria values increased somewhat in this group of rats, probably due to age related changes in kidney functions. At the end of the experiment proteinuria increases in the untreated and treated rats were compared to proteinuria values obtained in the metabolic control group rats.

Group II untreated animals with SPHN started to become proteinuric from 13 weeks after the induction of the disease and by 32 weeks 100% of the rats were proteinuric with an average of 350 mg/day proteinuria. Group II rats with SPHN pre- and post-treated with MICs started to become proteinuric also from 13 weeks after the induction the disease and by 32 weeks 50% of the rats were proteinuric with an average of 140 mg/day proteinuria.

Group IV rats with SPHN post-treated with MICs, just as group II and III rat became proteinuric from 13 weeks after the induction of the disease and by 32 weeks 80% of to the rats were proteinuric with an average of 220 mg/day proteinuria.

At the end of the experiment group II rats were 10× more, group III rats 4× more and group IV rats 6× more proteinuric then the metabolic controls.

Light microscopy: Kidney sections of metabolic control rats showed no morphological changes on H&E and Methenamine silver stained sections. Kidney sections of group II rats with SPHN staining for H&E showed increased glomerular cellularity and by the Methenamine silver stain prominent mesangial areas and thickened glomerular capillaries with silver positive projections on their outer circumferences. Group III rats with SPHN pre- and post-treated with MICs showed similar but less pronounced kidney lesions then group II rats and animals with proteinuria values below 100 mg/day showed evenly thin glomerular capillary-loops with occasional silver positive projections on their outer circumferences. Group IV rats with SPHN post-treated with MICs manifested kidney lesions somewhere in between findings observed in-group II and III rats.

Electron microscopy: Metabolic rat kidney section showed no ultrastructural abnormalities. Ultrathin kidney cortical sections of group II rats with SPHN revealed typical HN kidney lesions. There were small to large osmiophilic deposits on the epithelial aspect of the irregularly thickened GBM partially or completely surrounded by BM-like material. Foot-processes were fused in relation to the deposits and epithelial cell cytoplasm showed osmiophilic areas especially near the deposits. Group III rats with SPHN pre- and post-treated with MICs showed a mild form of HN. Mainly small osmiophilic deposits were present on the epithelial side of the GBM. Projections of the GBM were evident in many areas, but thickening of the GBM as a result of the projections did not result in multilayered trapping of deposits, which were observed on the kidney sections of group II rats. Foot-processes were fused in relation to deposits and epithelial cell cytoplasm showed osmiophilic areas opposite the deposits. Group N rats with SPHN post-treated with MICs showed a range of typical HN-kidney lesions. High proteinuric rats show more numerous deposits on their GBMs with additional typical changes while low proteinuric rats had fewer deposits on their GBMs just like group III rats.

Direct fluorescent antibody test results: Diffuse beaded deposition of rat IgG staining with intense fluorescence around the glomerular capillary-loops was observed on the kidney sections of group II rats at 8 weeks after the induction of the disease. In addition presence of rat IgG in one or all of these structures: the BB, TBM and BC was recorded on the kidney sections of seven rats. Pre- and post-treated rats in groups III and N had lower glomerular grade lesions and fewer sections stained the BB, TBM, and BC. Kidney sections of metabolic rats did not stain for rat IgG Mesangial regions of rat kidney sections stained for rat IgM with a similar fluorescent intensity and grades in all groups of rats, including metabolic controls.

At the end of the experiment at 32 weeks glomerular depositions of ICs staining for rat IgG were most advanced in the group II SPHN rats. The mildest glomerular lesions were still observed in group III and IV rats treated with MICs. In these animals lower glomerular grade lesions were found with fewer rat kidney sections staining the BB, TBM, or BC. Mesangial deposition of rat IgM was same as at 8 weeks in the kidneys of group I and II rats but considerably reduced in the treated group III and N rats. Faint beaded deposition of rat IgM was observed around the glomerular capillaries in most glomeruli of rats irrespective the groups they belonged to. Kidney sections were also stained at the end of the experiment for the presence of C5b-9. Glomerular capillaries of untreated group II rats stained strongly with a beaded pattern for C5b-9 while the glomeruli of most group III and IV rats stained with a faint beaded pattern of fluorescence.

Indirect fluorescent antibody test results: Progression of SPHN is maintained by presence of pathogenic autoantibodies in the circulation. Therefore periodic evaluation of circulating pathogenic and non-pathogenic autoantibodies can give us a good, idea, which phase (downward or upward trend) the untreated and treated rats are in their disease progression.

Group I. Metabolic control rats, during the experiment, had a low level of naturally occurring IgM autoantibodies in their circulation directed against the renal tubular BB regions of the proximal convoluted tubules.

In-group II SPHN untreated rats the average circulating IgG autoantibody level was high throughout the experiment, even at the end of the experiment. The IgM autoantibody level was below the IgG autoantibody level but somewhat above normal values. Five rats with low G/M ratios had lower IgG autoantibody and higher IgM autoantibody responses, indicating that at least in some of the rats a naturally occurring down-regulatory trend takes place aiming to terminate the disease process. However 5 rats with high G/M ratios had a continuously high pathogenic autoantibody response, showing no resolution.

Injection of an aqueous azo rKF3 antigen at 22 weeks three times at 5 day intervals increased the pathogenic autoantibody response in over 70% of the rats immediately and this increased response was still evident at the end of the experiment at 32 weeks in about 60% of the rats.

In group III SPUN pre- and post-treated rats with MICs the initial pathogenic autoantibody response by weeks 2, 7, and 8 was high, though in reality approximately 3× less then in group II animals and then after by week 12, 7× less and by week 16, 8× less. Subsequently, even after the repeated injections of the aqueous azo-rKF3 antigen at 22 weeks, the level of pathogenic autoantibody was low in the serum of every rat, indicating an excellent response in both low and high G/M ratio rats to the injected MICs. At the end of the experiment at seven months 90% of the rats had insignificantly low levels of circulating IgG autoantibodies and at eight months 70% of the rats. By 7 months, 5 rats and by 8 months 6 rats had no pathogenic autoantibodies in their circulation. These results show that increased levels of specific IgM autoantibodies can effectively take altered autoantigens out of the circulation. This effect was well demonstrated by the after 22 week results when repeated injections of an aqueous azo-rKF3 antigen only marginally increased the pathogenic autoantibodies for a short period of time only and then after decreased them even to 0 in 6 rats.

In group IV SPHN post-treated rats with MICs the initial pathogenic autoantibody response at 7 and 8 weeks was quite high and then after it started to decline quite dramatically, corresponding with increased presence of IgM autoantibodies in the circulation. On average the pathogenic IgG autoantibody level was low at the end of the experiment. At 32 weeks 90% of the rats had insignificantly low levels of IgG autoantibodies and 60% of the rats had no IgG autoantibodies in their sera. Repeated restimulation of group IV rats with aqueous azo-rKF3 antigen at 22 weeks only temporarily increased IgG antibody response and by the end of the experiment 6 rats were free of any pathogenic autoantibodies in the circulation. These results show that pathogenic autoantibody responses driven by modified self-antigens can be effectively brought under control by immune regulation during the course of the developing autoimmune disease.

Overall progression of autoimmune disease processes in treated and untreated rats: Overall progression of autoimmune disease process is illustrated in Figure X. Combined results of G/M ratios are plotted for group II untreated and groups III and IV rats treated with MICs. Pre- and post-treated rats with MICs in group III had by far the least progression of their autoimmune disease processes and animals treated from 7 weeks had greatly reduced disease progressions as compared to untreated group II rats' disease progressions. Looking at the overall progression of the disease during the entire experiment, group III rats were 11× better off and group IV rats 2.5× better off then group II rats. It seems during the first 8 weeks downregulation of pathogenic autoantibody responses by MICs was not as effective as it was later into the disease in-group III animals. However, by week 12 down-regulatory response to MIC treatments was most effective in both group III and IV animals. Restimulation with aqueous azo rKF3 antigen at 22 weeks, group II rats responded with a significant and continuous production of pathogenic autoantibodies, while group III and IV rats did not, indicating excellent down-regulatory effects by MICs. At the end of the experiment at 32 weeks in group II rats the average G/M ratio was 8 revealing a progressive autoimmune disease to continue, while in groups III and IV rats the average G/M ratios were 0.12 and 0.375 respectively showing downregulation of pathogenic autoantibodies responses.

Discussion: These experiment demonstrate that the methods and compositions of the invention can be used to ameliorate autoimmune disease. The compositions of the invention comprise ICs at slight antigen excess. In the experiments described above, the exemplary compositions of the invention comprise nephritogenic antigen and homologous IgM antibody directed against it. Injecting these ICs (designated as MICs) increased the level of circulating IgM autoantibodies by specifically stimulating the CD5+ B cell lines. The increased level of IgM autoantibodies were able to remove the circulating chemically altered and unaltered nephritogenic autoantigens released from the renal proximal convoluted tubules and thereby prevented two major events to continue which could significantly contribute to chronic progression of the autoimmune disease. First, the methods and compositions of the invention assisted in the removal of the altered self-antigen and thereby prevented pathogenic IgG autoantibody production, and secondly, the methods and compositions of the invention assisted in the removal of the unaltered nephritogenic autoantigen released from the proximal convoluted tubules and prevented further fixation and deposition of this autoantigen in the glomeruli to free IgG autoantibody sites.

These experiments demonstrate that the methods and compositions of the invention are effective for the antigen-specific downregulation of pathogenic autoantibody responses. Methods and compositions of the invention, comprise a novel vaccination technique employing antibody information transfer by administering the compositions of the invention (e.g., by the injected MICs) to treat numerous autoimmune diseases of man. The treatment regiments of the invention can specifically boost the level of naturally occurring IgM autoantibodies in the circulation and will be able to terminate pathogenic autoantibody responses even during the acute or chronic phases of an autoimmune disease without causing side effects.

Table 4 shows kidney biopsies at 8 and 32 weeks staining by direct fluorescent antibody test for rat IgG and Rat IgM. Average values are given within the groups. Fluorescent intensity and grade of glomerular lesions of SHIN untreated (Group II) and variously treated (Group III and IV) rats are shown. Metabolic controls (Group I) are also graded. Each group had 10 rats.

TABLE 4

| Anti- rat IgG | | | | | | |
|---|---|---|---|---|---|---|
| | | Grade | | | | |
| | | increase due | TBM, | Anti- rat IgM | | |
| Glomerular | Glomerular | to TBM, BB, | BB, BC | Mesangium | Mesangium | Glomerular loop |
| Intensity | Grade | BC staining+ | presence* | Intensity | Grade | presence |

8 weeks

| Gr. I Metabolic Controls | 0 | 0 | 0 | | 0 | 2 | 0.6 | 10/10 |
|---|---|---|---|---|---|---|---|---|
| Gr. II SPHN | 3.2 | 1.96 | 2.4 | (9) | 7 | 2.9 | 0.9 | 10/10 |
| Gr. III SPHN Pre-/Post- Tx w/MICs | 2.7 | 0.66 | 0.7 | (10) | 2 | 2.5 | 0.6 | 9/10 |
| Gr. IV SPHN Post- Tx w/MICs | 3.4 | 1.76 | 2 | (7) | 4 | 2.9 | 0.7 | 9/10 |

32 weeks

| Gr. I Metabolic Controls | 0 | 0 | 0 | | 0 | 3.2 | 0.8 | 8/10 |
|---|---|---|---|---|---|---|---|---|
| Gr. II SPHN | 4 | 3 | 3.25 | (1) | 5 | 3 | 0.8 | 5/10 |
| Gr. III SPHN Pre-/Post- Tx w/MICs | 3.2 | 2 | 2.1 | (4) | 3 | 2.2 | 0.4 | 6/10 |
| Gr. IV SPHN Post- Tx w/MICs | 3.8 | 2.3 | 2.5 | (3) | 4 | 2.4 | 0.4 | 4/10 |

Abbreviations: BB: brush border; BC: Bowman's capsule; MICs: immune complex M; SPHN: slowly progressive Heymann nephritis, TBM: tubular basement membrane; Tx: treated; w: with
*member of rat kidneys staining one or more of these structures
+member of rat kidneys below grade 2 glomerular lesions (in brackets).

A number of embodiments of the invention have been described. Nevertheless, it will be understood that various modifications may be made without departing from the spirit and scope of the invention. Accordingly, other embodiments are within the scope of the following claims.

1. A method for increasing the levels of an autoantigen-specific IgM antibody in a mammal, or decreasing the levels of a circulating autoantigen in a mammal, comprising the following steps:
(a) providing a composition comprising an unmodified autoantigen and an antigen-specific multi-valent antibody, wherein the multi-valent antibody is specific for the autoantigen and is native to the mammal or is non-immunogenic to the mammal, and the autoantigen is present in the composition in molar excess to the multi-valent antibody; and
(b) administering to the mammal an amount of the composition sufficient to increase the levels of the antigen-specific IgM antibody in the individual.

2-3. (canceled)

4. The method of claim 1, wherein the mammal is a human.

5. The method of claim 1, wherein the multi-valent antibody comprises an IgM, an isolated antibody, a synthetically generated antibody, a recombinantly generated antibody, a humanized antibody or a human antibody generated in a transgenic mouse.

6-9. (canceled)

10. The method of claim 1, wherein in making the composition comprising the autoantigen and the antigen-specific multi-valent antibody (a) the unmodified autoantigen is mixed with the multi-valent antibody immediately before administration, or between about 1 minute and two hours before administration, or between about 5 minutes and one hour before administration, or between about 10 minutes and 30 minutes before administration, or (b) the unmodified autoantigen is mixed with the multi-valent antibody and the mixture is freeze-dried, or the freeze-dried mixture is reconstituted in a formulation for administration at the time of administration.

11-18. (canceled)

19. The method of claim 1, wherein the autoantigen comprises a purified autoantigen; a recombinant or synthetic polypeptide; a soluble antigen; a particulate antigen; a small molecular weight antigen; antigen having a molecular weight of between about 0.1 to 10 kd or about 0.5 to 5 kd; a large molecular weight antigen; an antigen having a molecular weight of between about 5 to 50 kd or about 10 to 25 kd; an autoantigen involved in an autoimmune response; a kidney tubular nephritogenic antigen, a glomerular nephritogenic antigen, an endometrial repro-EN-1.0 antigen, an endometrial IB1 antigen, glutamic acid decarboxylase, nucleolar ASE-1 antigen, Ro/SSA, La/SSB, nRNP, Sm, transaldolase, myelin basic protein, 70 kD mitochondrial biliary autoantigen, human cartilage glycoprotein 39, human Sp17 protein, or a human placental Hp-8; a subcellular fraction, a cell, a tissue or an organ involved in the autoimmune response.

20-81. (canceled)

82. A method for increasing the levels of an antigen-specific IgG antibody in a mammal, or decreasing the levels of a circulating antigen in a mammal, comprising the following steps:
(a) providing a composition comprising a modified antigen and an antigen-specific bi-valent antibody, wherein the bi-valent antibody is specific for the antigen and is native to the mammal or is non-immunogenic to the mammal, and the modified antigen is present in the composition in molar excess to the bi-valent antibody; and
(b) administering to the mammal an amount of the composition sufficient to increase the levels of the antigen-specific IgG antibody in the individual.

83-84. (canceled)

85. The method of claim 82, wherein the mammal is a human.

86. The method of claim 82, wherein the bi-valent antibody comprises: an IgG; an isolated antibody, a synthetic antibody or a recombinantly generated antibody; a humanized antibody; or, a human antibody generated in a transgenic mouse.

87-130. (canceled)

131. A pharmaceutical composition comprising (i) a modified antigen and an antigen-specific bi-valent antibody, wherein the bi-valent antibody is specific for the antigen and is native to the mammal or is non-immunogenic to the mammal, and the modified antigen is present in the composition in molar excess to the bi-valent antibody, and (ii) a pharmaceutically acceptable excipient.

132. The pharmaceutical composition of claim 131, wherein the bi-valent antibody comprises: an IgG; or, an isolated antibody, a synthetic antibody or a recombinantly generated antibody; a humanized antibody; or, a human antibody generated in a transgenic mouse.

133-136. (canceled)

137. The pharmaceutical composition of claim 131, wherein in making the composition comprising the modified antigen and the antigen-specific bi-valent antibody (a) the modified antigen is mixed with: the bi-valent antibody immediately before administration; or, the bi-valent antibody between about 1 minute and two hours before administration; or, the bi-valent antibody between about 10 minutes and one hour before administration; or, the bi-valent antibody between about 30 minutes and one hour before administration; or, the bi-valent antibody and the mixture is freeze-dried; or, (b) the freeze-dried mixture is reconstituted in a formulation for administration at the time of administration, and optionally the freeze-dried mixture is stored at a temperature of between about −20° C. and 4° C., or the freeze-dried mixture is reconstituted in an aqueous formulation, and optionally the aqueous formulation comprises sterile distilled water or buffered saline.

138-145. (canceled)

146. The pharmaceutical composition of claim 131, wherein the antigen: comprises a purified antigen; or, comprises a recombinant or synthetic polypeptide; or comprises a soluble antigen; or, comprises a particulate antigen; or, comprises a small molecular weight antigen or a large molecular weight antigen; or, comprises the antigen has a molecular weight of is between about 0.1 to 10 kd or about 0.5 to 5 kd, or, between about 5 to 50 kd or about 10 to 25 kd; or, comprises a cancer-specific antigen or an antigen specific for a hyperplastic cell or tissue; or, a foreign antigen; or, comprises a bacterial antigen, a viral antigen, a fungal antigen, a yeast antigen or a protozoan antigen; or, comprises or is derived from a subcellular fraction, a cell, a tissue, an organ, a subcellular fraction, a cell or tissue homogenate or a cell, tissue or organ extract; or, comprises or is derived from a melanoma, prostate cancer, thyroid cancer, pancreatic cancer, liver cancer, breast cancer, lung cancer or stomach cancer antigen; or comprises or is derived from an antigen from a pathogen or infectious disease agent; or, comprises or is derived from a bacterial antigen, a viral antigen or an antigen from a protozoan; or, comprises or is derived from Staphylococcus, Streptococcus, E. coli, flu virus, hepatitis A, B or C, or malaria.

147-173. (canceled)
